(19) United States
(12) Patent Application Publication
(10) Pub. No.: US 2024/0148735 A1
(43) Pub. Date: May 9, 2024

(54) METHODS OF TREATING HEART FAILURE WITH VIBEGRON

(71) Applicant: UROVANT SCIENCES GMBH, Zug (CH)

(72) Inventor: Jihao ZHOU, Irvine, CA (US)

(21) Appl. No.: 18/546,585

(22) PCT Filed: Feb. 16, 2022
(86) PCT No.: PCT/IB2022/051393

§ 371 Date: Aug. 15, 2023

Related U.S. Application Data
(60) Provisional application No. 63/150,044, filed on Feb. 16, 2021.
(60) Provisional application No. 63/294,891, filed on Dec. 30, 2021.

(51) Int. Cl. A61K 31/519; A61K 45/06; A61P 9/04

(52) U.S. Cl. CPC A61K 31/519; A61K 45/06; A61P 9/04

(57) ABSTRACT

The present disclosure provides methods of treating or preventing heart failure and/or one or more symptoms thereof, the methods comprising administering to a subject in need thereof a therapeutically effective amount of vibegron.

### CROSS-REFERENCE TO RELATED APPLICATIONS

This application claims the priority benefit of U.S. Provisional Application No. 63/150,044, filed Feb. 16, 2021, and U.S. Provisional Application No. 63/294,891, filed Dec. 30, 2021, both of which are incorporated by reference herein in their entireties.

### BACKGROUND

Heart failure is a common, chronic, costly, serious, and potentially fatal condition where the heart is unable to pump sufficiently to maintain blood flow to meet the body tissue's needs for metabolism. In general terms, based on the severity of a patient's symptoms, heart failure is often classified using the New York Heart Association's (NYHA) classification, which stages the disease based on the functional status of the patient. Heart failure that is specific to the left ventricle is classified as either heart failure with reduced ejection fraction (HFrEF) or heart failure with preserved ejection fraction (HFpEF), based on whether the ability of the left ventricle to contract or to relax is affected.

As the beta-3 adrenergic receptor (β3-AR) has been identified in the myocardium, it is a potential target for new therapeutic approaches for heart disease (see, for example, Cannavo, A. et al., Cardiovasc. Pharmacol. 2017, 69, 71-78). Some studies have shown that β₃-AR is upregulated in human heart failure and animal models (see Germack et al. J. Pharmacol. Exp. Ther. 316 (2006), and Moniotte et al., Circulation 103 (2001)), causing some to hypothesize that the negative inotropic effects of β₃-AR are detrimental and that diminishing β₃-AR activity could be beneficial in the treatment of heart failure. See Rozec et al. Arch. Mal. Coeur Vaiss. 96 (2003) and Moniotte et al. Cardiovasc. Drug Rev. 20 (2002). Some studies have demonstrated that β₃-AR stimulation induces a decrease in human cardiac contractility through activation of a nitric oxide synthase (NOS) pathway (see, for example, Gauthier et al. J. Clin. Invest. 102 (1998)). Thus, treatment with a β₃-AR antagonist may serve to deactivate the NOS pathway, and, as a result, increase cardiac contractility. Therapies, however, have targeted both (β3-AR antagonism and agonism.

For example, beta-3 adrenergic receptor antagonists have demonstrated a trend toward improved cardiac contractility with increased dosage in rats with induced HFrEF (see, for example, U.S. Pat. No. 10,470,797). Beta-3 adrenergic receptor agonists have also been investigated as potential heart failure therapies. Two recent clinical trials refer to the use of β3-AR agonist mirabegron in heart failure. In the BEAT-HF trial, mirabegron was administered to subjects with New York Heart Association (NYHA) class II-III HF and a left ventricular ejection fraction (LVEF) <40% as determined by echocardiography at screening. The BEAT-HF trial failed to meet the primary endpoint (change in LVEF). See, for example, Bundgaard, H. et al., European J. of Heart Failure, 2017; 19: 566-575.

In the ongoing Beta3-LVH trial, the effect of mirabegron in patients with increased left ventricular mass index by echocardiography or end-diastolic wall thickness ≥13 mm in at least one wall segment is being examined. In contrast to the BEAT-HF trial, the Beta3-LVH trial is examining the use of mirabegron in HFpEF (see, for example, Pouleur, A. C. et al., ESC Heart Failure, 2018; 5: 831-842). Treatment modalities for HFpEF have so far been with only limited success.

Despite these previous studies of β₃-AR and heart failure, it remains unknown what modulation of the adrenergic receptor will benefit patients suffering from or at risk of heart failure. And given the growing public health burden associated with heart failure (affecting 2-3% of adults in developed countries, see, for example, Benjamin, et al., Circulation. 2017 Mar. 7; 135(10): e146—e603), there remains a significant medical need for effective treatment of heart failure.

### BRIEF DESCRIPTION OF THE DRAWINGS

FIG. 1 shows the response-concentration curve for vibegron and mirabegron at β₃-adrenergic receptors relative to the full agonist isoproterenol.

FIG. 2A shows the measured percentage of left ventricle fractional shortening in mice with induced myocardial infarction after 4 weeks of treatment with vibegron.

FIG. 2B shows the measured percentage of left ventricle ejection fraction in mice with induced myocardial infarction after 4 weeks of treatment with vibegron.

FIG. 3A shows the measured stroke volume in microliters (μL) in mice with induced myocardial infarction after 4 weeks of treatment with vibegron.

FIG. 3B shows the measured cardiac output in milliliters per minute (mL/min) in mice with induced myocardial infarction after 4 weeks of treatment with vibegron.

FIG. 4A shows the change in the maximum rate of left ventricle contraction pressure (mm Hg/s) with increasing doses of isoproterenol in mice with induced myocardial infarction after 4 weeks of treatment with vibegron.

FIG. 4B shows the change in the minimum rate of left ventricle relaxation pressure (mm Hg/s) with increasing doses of isoproterenol in mice with induced myocardial infarction after 4 weeks of treatment with vibegron.

FIG. 4C shows the change in heart rate (bpm) with increasing doses of isoproterenol in mice with induced myocardial infarction after 4 weeks of treatment with vibegron.

FIGS. 5A to 5F are histology sections showing the effects of 4 weeks of treatment with varying doses of vibegron on mouse hearts with induced myocardial infarction.

FIG. 5G shows the measured left ventricle lumen area in mice with induced myocardial infarction after 4 weeks of treatment with vibegron.

FIG. 5H shows the measured percent (%) of infarcted myocardium in mice with induced myocardial infarction after 4 weeks of treatment with vibegron.

FIG. 6 shows a graph of vibegron plasma titers (ng/mL) for mice with induced myocardial infarction after 4 weeks of treatment with vibegron.

### SUMMARY

In certain aspects, the present disclosure provides a method for treating heart failure with preserved ejection fraction (HFpEF), one or more symptoms thereof, or both in a subject in need thereof, the method comprising administering a therapeutically effective amount of vibegron to the subject. In some aspects, the present disclosure provides a method for preventing heart failure with preserved ejection fraction (HFpEF), the method comprising administering a therapeutically effective amount of vibegron to the subject. In some aspects, the subject has a left ventricular ejection fraction of greater than about 40%. In some aspects, the subject has a left ventricular ejection fraction of greater than about 50%. In certain aspects, the treating comprises halting the progression of HFpEF, relieving one or more symptoms associated with HFpEF, or both.

In certain aspects, the present disclosure provides a method for treating heart failure with reduced ejection fraction (HFrEF), one or more symptoms thereof, or both, in a subject in need thereof, the method comprising administering a therapeutically effective amount of vibegron to the subject. In some aspects, the present disclosure provides a method for preventing heart failure with reduced ejection fraction (HFrEF), the method comprising administering a therapeutically effective amount of vibegron to the subject. In some aspects, the subject has a left ventricular ejection fraction less than about 50%. In some aspects, the subject has a left ventricular ejection fraction of less than about 40%. In some aspects, the treating comprises halting the progression of HFrEF, relieving one or more symptoms associated with HFrEF, or both.

In certain aspects, the present disclosure provides a method of agonizing β₃-adrenergic receptor and antagonizing β₁-adrenergic receptor, β₂-adrenergic receptor, or both β₁-adrenergic receptor and β₂-adrenergic receptor in a subject in need thereof, the method comprising administering 1) a therapeutically effective amount of vibegron to the subject to agonize β₃-adrenergic receptor and 2) a therapeutically effective amount of a therapeutic agent that antagonizes β₁-adrenergic receptor, that antagonizes β₂-adrenergic receptor, or that antagonizes both β₁-adrenergic receptor and β₂-adrenergic receptor. In some aspects, the subject suffers from heart failure, one or more symptoms thereof, or both. In some aspects, the subject has heart failure with preserved ejection fraction (HFpEF), one or more symptoms thereof, or both. In some aspects, the subject has heart failure with reduced ejection fraction (HFrEF), one or more symptoms thereof, or both. In some aspects, the subject has heart failure with mid-range ejection fraction (HFmrEF), one or more symptoms thereof, or both. In some aspects, agonizing β₃-adrenergic receptor comprises agonizing β₃-adrenergic receptor in the cardiovascular system of the subject. In some aspects, agonizing β₃-adrenergic receptor comprises agonizing β₃-adrenergic receptor in the myocardium of the subject.

In certain aspects of the methods described herein, the therapeutically effective amount of vibegron is from about 10 mg to about 450 mg per day. In certain aspects, the therapeutically effective amount of vibegron is from about 50 mg to about 450 mg per day. In certain aspects, the therapeutically effective amount of vibegron is from about 50 mg to about 300 mg per day. In certain aspects, the therapeutically effective amount of vibegron is from about 90 mg to about 450 mg per day. In some aspects, the therapeutically effective amount of vibegron is from about 100 mg to about 400 mg per day. In some aspects, the therapeutically effective amount of vibegron is from about 100 mg to about 200 mg per day.

In some aspects of the methods described herein, the method further comprises administering to the subject a second therapeutic agent. In some aspects, the second therapeutic agent is selected from an antihyperlipoproteinemic agent (for example, HMG-CoA reductase inhibitors such as atorvastatin, fluvastatin, lovastatin, pravastatin, rosuvastatin, or simvastatin, or other agents such as ezetimibe, fibric acid derivatives, niacin, or bile acid sequestrants, such as cholestyramine, colestipol, or colesevelam), an antiarteriosclerotic agent, an antithrombotic/fibrinolytic agent, a blood coagulant (for example aprotinin, tranexamic acid, epsilon-aminocaproic acid, or aminomethylbenzoic acid), an antiarrhythmic agent (for example, amiodarone, flecainide, ibutilide, lidocaine, procainamide, propafenone, quinidine, or tocainide), an antihypertensive agent (for example furosemide, bumetanide, amiloridhydralazine, minoxidil, spironolactone, clonidine, or methyldopa), a vasopressor (for example, norepinephrine, epinephrine, vasopressin, dopamine, phenylephrine, or dobutamine), a diuretic (for example, chlorthalidone, hydrochorothiazide, metolazone, indapamide, bumetanide, ethacrynic acid, or furosemide), a statin (for example, atorvastatin, fluvastatin, lovastatin, pravastatin, rosuvastatin, or simvastatin), an anti-platelet agent (for example, aspirin, clopidogrel, ticlopidine, ticagrelor, prasugrel, or cangrelor), an SGLT2 inhibitor (for example, canagliflozin, dapagliflozin, orempagliflozin), a treatment agent for congestive heart failure, an antianginal agent (for example, a nitrate such as isosorbide dinitrate, isosorbide mononitrate, or nitroglycerin; a calcium antagonist such as diltiazem, nifedipine, nimodipine, or verapamil; a beta blocker such as atenolol, pindolol, propranolol, or metoprolol, or ranolazine), an antibacterial agent, a renin-angiotensin system (RAS) blocker (for example, captopril, imidapril, zofenopril, candesartan, delapril, telmisartan, aliskiren, or moexipril), a mineralocorticoid receptor antagonist (MRA) (for example, spironolactone or eplerenone), an ACE inhibitor (for example benazepril, captopril, enalapril, fosinopril, lisinopril, moexipril, perindopril, or quinapril), an angiotensin receptor blocker (for example, azilsartan, candesartan, eprosartan, irbesartan, losartan, olmesartan, telmisartan, or valsartan), a neprolysin inhibitor antagonist (for example, sacubitril/valsartan), an alpha-2 receptor agonist (for example, guanabenz, guanfacine, clonidine, tizanidine, medetomidine, or dexmedetomidine), and a combination thereof.

In certain aspects of the methods described herein, the vibegron is administered orally. In some aspects, the vibegron is administered parenterally. In some aspects, the vibegron is administered intravenously.

In certain aspects of the methods described herein, the subject receiving vibegron experiences a smaller maximum mean change in systolic blood pressure than a subject receiving a therapeutically effective amount of mirabegron. In some aspects, the subject receiving vibegron experiences an increase in maximum mean change in systolic blood pressure of from about 1.5 mm Hg to about 4.0 mm Hg smaller than a subject receiving a therapeutically effective amount of mirabegron. In some aspects, the subject receiving vibegron experiences a smaller mean 24 hour change in systolic blood pressure than a subject receiving a therapeutically effective amount of mirabegron. In some aspects, the subject receiving vibegron experiences an increase in mean 24 hour change in systolic blood pressure of from about 1.0 mm Hg to about 10.0 mm Hg smaller than a subject receiving a therapeutically effective amount of mirabegron. In some aspects, the subject receiving vibegron experiences a smaller maximum increase in heart rate than a subject receiving a therapeutically effective amount of mirabegron. In some aspects, the subject receiving vibegron experiences an increase in maximum mean increase in heart rate that is from about 2 bpm to about 14 bpm smaller than a subject receiving a therapeutically effective amount of mirabegron. In some aspects, the therapeutically effective amount of mirabegron is from about 50 mg to about 300 mg.

In certain aspects of the methods described herein, the vibegron is administered once per day. In some aspects, the vibegron is administered with a meal. In some aspects, the vibegron is administered without a meal. In certain aspects, the vibegron is administered as a free base. In some aspects, the vibegron is administered as a pharmaceutically acceptable salt thereof.

In certain aspects of the methods described herein, the subject's contractile function is increased or improved relative to a subject who has not received vibegron or as compared to the subject's contractile function before receiving vibegron.

In certain aspects of the methods described herein, the increase or improvement in the subject's contractile function is measured by fractional shortening. In some aspects the subject's fractional shortening is increased relative to a subject who has not received vibegron. In some aspects, the subject's fractional shortening is increased by about 2% to about 15% relative to a subject who has not received vibegron. In some aspects, the subject's fractional shortening is increased by about 5% to about 10% more than a subject who has not received vibegron.

In certain aspects of the methods described herein, the increase or improvement in the subject's contractile function is measured by ejection fraction. In some aspects, the subject's ejection fraction is increased relative to a subject who has not received vibegron. In some aspects, the subject's ejection fraction is increased by about 2% to about 20% more than a subject who has not received vibegron. In some aspects, the subject's ejection fraction is increased by about 10% to about 20% more than a subject who has not received vibegron.

In certain aspects of the methods described herein, the blood volume in the subject's left ventricle is increased or improved relative to a subject who has not received vibegron or as compared to the subject's blood volume before receiving vibegron.

In certain aspects of the methods described herein, the increase or improvement in the subject's blood volume is measured by stroke volume in the subject's left ventricle. In some aspects, stroke volume in the subject's left ventricle is increased relative to a subject who has not received vibegron. In some aspects, the stroke volume in the subject's left ventricle is increased by about 25% to about 75% relative to a subject who has not received vibegron. In some aspects, the stroke volume in the subject's left ventricle is increased by about 30% to about 65% relative to a subject who has not received vibegron.

In certain aspects of the methods described herein, the increase or improvement in the subject's blood volume is measured by cardiac output. In some aspects, the subject's cardiac output is increased relative to a subject who has not received vibegron. In some aspects, the subject's cardiac output is increased by about 25% to about 75% relative to a subject who has not received vibegron. In some aspects, the subject's cardiac output is increased by about 30% to about 65% relative to a subject who has not received vibegron.

### DETAILED DESCRIPTION

Vibegron, (6S)-N-[4-[[(2S,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide, is a potent and highly selective beta-3 adrenergic receptor agonist demonstrating >9,000 fold selectivity for activation of β₃-AR over β₂-AR and β₁-AR in cell based in vitro assays. See Edmondson et al., J. Med. Chem. 59:609-623 (2016).

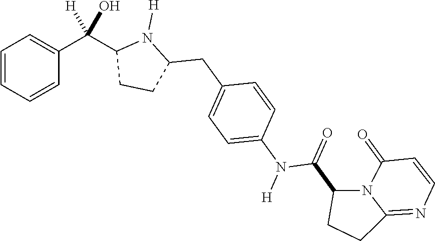

Vibegron is disclosed as a β₃-AR agonist in U.S. Pat. Nos. 8,399,480, 8,653,260, and 8,247,415. Synthetic methods for preparing vibegron are disclosed in United States Publication Nos. US 2017/0145014, US 2015/0087832, US 2016/0176884, and US 2014/0242645. All of the cited publications are herein incorporated by reference in their entireties.

While vibegron has been approved for use in the treatment of overactive bladder, its selectivity against β₃-AR, particularly in comparison to other agonists such as mirabegron, supports that it can be effective in the treatment of heart failure. β₃-AR is proposed to have multiple functions within the cardiovascular system, at least one of which provides a mostly protective role for the cardiovascular system. For example, use of some β₁-AR-blockers, used to treat HFrEF, has been associated with improvement of the β₃-AR signaling pathway. See Cannavo and Koch, J Cardiovasc Pharmacol 2017; 69:71-78. Thus, direct agonism of the β₃-AR with highly-selective vibegron, or dual agonism of β₃-AR and antagonism of β₁-AR with highly-selective vibegron and a beta blocker, should improve the protective role of β₃-AR within the cardiovascular system.

Previously it has been reported that nebivolol, both a β₁-AR antagonist and a β₃-AR agonist, has shown some success in treating heart failure in elderly patients, including a subgroup with HFpEF (see, for example, Eur. Heart J. 2005 February; 26(3): 215-225). Without being bound by a particular theory, vibegron, which is a highly selective β₃-AR agonist and not a β₁-AR antagonist, could be even more effective against heart failure.

In certain aspects, the present disclosure provides a method of agonizing β₃-adrenergic receptor in a subject in need thereof, the method comprising administering a therapeutically effective amount of vibegron to the subject. In certain aspects, the present disclosure provides a method of agonizing β₃-adrenergic receptor and antagonizing β₁-adrenergic receptor in a subject in need thereof, the method comprising administering 1) a therapeutically effective amount of vibegron to the subject to agonize β₃-adrenergic receptor and 2) a therapeutically effective amount of a therapeutic agent that antagonizes β₁-adrenergic receptor. In certain aspects, the present disclosure provides a method of agonizing β₃-adrenergic receptor and antagonizing β₂-adrenergic receptor in a subject in need thereof, the method comprising administering 1) a therapeutically effective amount of vibegron to the subject to agonize β₃-adrenergic receptor and 2) a therapeutically effective amount of a therapeutic agent that antagonizes β₂-adrenergic receptor. In certain aspects, the present disclosure provides a method of agonizing β₃-adrenergic receptor and antagonizing both a β₁- and a β₂-adrenergic receptor in a subject in need thereof, the method comprising administering 1) a therapeutically effective amount of vibegron to the subject to agonize β₃-adrenergic receptor and 2) a therapeutically effective amount of one or more therapeutic agents that antagonize a β₁- and a β₂-adrenergic receptor.

### Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about” as used in connection with a numerical value throughout the specification and the claims denotes an interval of accuracy, familiar and acceptable to a person skilled in the art. In certain aspects, such interval of accuracy is ±10%. In other aspects, such interval of accuracy is ±5%.

The term “agonize” as used herein refers to when a compound binds to a receptor and produces a response that is, or is similar to, the receptor's intended response.

The term “antagonize” as used herein refers to when a compound binds to a receptor and partially or completely stops the receptor from producing its intended response.

As used herein, the terms “subject” and “subjects” are synonymous with and can be used interchangeably with “patient” and “patients.”

The term “free base” as used herein refers to a basic chemical compound itself, not in the form of a salt. For example, vibegron free base refers to (6S)-N-[4-[[(2S,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide.

The term “pharmaceutically acceptable salt” means those salts of compounds that are safe and effective for use in subjects and that possess the desired biological activity.

Pharmaceutically acceptable salts of a basic compound can be salts of organic or inorganic acids. In some aspects, the organic and inorganic acids include but are not limited to hydrochloric acid, hydrobromic acid, sulfuric acid, phosphoric acid, citric acid, maleic acid, mandelic acid, succinic acid and methanesulfonic acid. (see generally, J. Pharm. Sci., 66, 2 (1977), which is incorporated herein by reference in its entirety).

The term “ejection fraction” refers to a measurement, expressed as a percentage, of how much blood the left ventricle pumps out with each heart contraction (referred to as systole). Typically, and in some aspects, a normal ejection fraction for an adult over 20 years of age is from about 53% to about 73%.

The terms “heart failure with preserved ejection fraction” and “HFpEF” refer to a condition where the left ventricle is not able to fill properly with blood during the diastolic (i.e., filling) phase resulting in a decreased amount of blood pumped out of the heart. HFpEF can be characterized by a peripheral insult, such as commonly associated with comorbidities) that secondarily causes myocardial dysfunction. Left ventricular hypertrophy caused by high blood pressure (hypertension) is a common example. In certain aspects, subjects with HFpEF have an ejection fraction of greater than about 50%. In certain aspects, subjects with HFpEF have an ejection fraction of ≥50%. In some aspects, subjects with HFpEF have an ejection fraction of greater than about 40%. In some aspects, subjects with HFpEF have an ejection fraction of ≥40%.

The terms “heart failure with reduced ejection fraction” and “HFrEF” refer to a condition where the left ventricle does not squeeze forcefully enough during the systolic (i.e., ejecting) phase resulting in a decreased amount of blood pumped out of the heart. HFrEF can be characterized by primary myocardial injury (e.g., myocardial infarction, viral cardiomyopathy, genetic abnormality, cardiotoxicity). In certain aspects, subjects with HFrEF have an ejection fraction of less than about 50%. In certain aspects, subjects with HFrEF have an ejection fraction of <50%. In some aspects, subjects with HFrEF have an ejection fraction of less than about 40%. In some aspects, subjects with HFrEF have an ejection fraction of <40%. In some aspects, subjects with moderate to severe HFrEF have an ejection fraction of <35%.

In certain aspects, subjects with an ejection fraction between about 40% and about 50% can be referred to as having mid-range ejection fraction (“HFmrEF”, see, for example, Andronic, A. et al., Maedica, 2016, 11, 320-324). In some aspects, the present disclosure provides a method for treating or preventing heart failure with HFmrEf, one or more symptoms thereof, or both in a subject in need thereof, the method comprising administering a therapeutically effective amount of vibegron to the subject.

The term “fractional shortening” refers to a measurement, expressed as a percentage, of change of left ventricle diameter that reflects, for example, left ventricle wall movement during systole and diastole.

The term “stroke volume” refers to a measurement, expressed as a unit of volume (e.g., liters), of the volume of blood pumped out of the left ventricle at systole or during each systolic cardiac contraction.

The term “cardiac output” refers to a measurement, expressed as a unit of volume (e.g., liters) per minute, of the total volume of blood pumped out of the left ventricle per minute.

The term “therapeutically effective amount” refers to an amount that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. When applied to an individual active ingredient, administered alone, the term refers to that ingredient alone. When applied in combination, the term refers to the combined amounts of the active ingredients that result in the therapeutic effect, whether administered in combination, serially, or simultaneously. An effective amount can be administered in one dosage or can be divided into multiple dosages, the total of such dosages being the effective amount. For example, an effective amount can be provided in two separate administrations over a period of time that, in aggregate, provide the effective amount of the formulation.

As used herein, the terms “treated,” “treating,” or “treatment” refer to partially or completely alleviating, ameliorating, improving, managing, relieving, delaying onset of, inhibiting progression of, reducing severity of, reducing incidence of one or more symptoms (including, but not limited to, fatigue or reduced exercise capacity) or features of heart failure, or any combination thereof. In certain aspects, the term “treating” refers to relieving one or more symptoms associated with HFrEF. In some aspects, the term “treating” refers to relieving one or more symptoms associated with HFpEF. In some aspects, the term “treating” refers to halting the progression of HFrEF. In some aspects, the term “treating” refers to halting the progression of HFpEF. In some aspects, the term “treating” refers to a combination of relieving one or more symptoms of heart failure and halting the progression of HFrEF. In some aspects, the term “treating” refers to a combination of relieving one or more symptoms of heart failure and halting the progression of HFpEF. In some aspects, the term “treating” refers to reversing structural remodeling of the heart.

In general, the term “treatment” refers to countering the effects caused as a result of the disease or pathological condition of interest in a subject including (i) inhibiting the progress of the disease or pathological condition, in other words, slowing or stopping the development or progression thereof, or one or more symptoms of such disorder or condition; (ii) relieving the disease or pathological condition, in other words, causing said disease or pathological condition, or the symptoms thereof, to regress; (iii) stabilizing the disease or pathological condition or one or more symptoms of such disorder or condition, (iv) reversing the disease or pathological condition or one or more symptoms of such disorder or condition to a normal state, (v) preventing the disease or pathological condition or one or more symptoms of such disorder or condition, and (vi) any combination thereof.

As used herein, the term “preventing” refers to partially or completely delaying onset of heart failure; partially or completely delaying onset of one or more symptoms, features, or clinical manifestations of heart failure (including, but not limited to, fatigue or reduced exercise capacity); partially or completely delaying onset of one or more symptoms, features, or manifestations of heart failure (including, but not limited to, fatigue or reduced exercise capacity); partially or completely delaying progression from heart failure; and/or decreasing the risk of developing pathology associated with heart failure.

As used herein, the term “onset of action” refers to the duration of time it takes for a drug's effects to come to prominence upon administration.

### Methods of Treatment

In certain aspects, the present disclosure provides methods of treating or preventing heart failure with preserved ejection fraction (HFpEF) and/or one or more symptoms thereof in a subject in need thereof. In certain aspects, the subject has a left ventricular ejection fraction of greater than about 50%. In certain aspects, the subject has a left ventricular ejection fraction of greater than about 45%. In certain aspects, the subject has a left ventricular ejection fraction of greater than about 40%. In some aspects, the subject has a left ventricular ejection fraction of greater than about 40%, greater than about 41%, greater than about 42%, greater than about 43%, greater than about 44%, greater than about 45%, greater than about 46%, greater than about 46%, greater than about 48%, greater than about 49%, or greater than about 50%. Preferably, the subject has a left ventricular ejection fraction of greater than 50%.

In certain aspects, the present disclosure provides methods of treating or preventing heart failure with reduced ejection fraction (HFrEF) and/or one or more symptoms thereof in a subject in need thereof. In certain aspects, the subject has a left ventricular ejection fraction of less than about 50%. In certain aspects, the subject has a left ventricular ejection fraction of less than about 45%. In certain aspects, the subject has a left ventricular ejection fraction of less than about 40%. In some aspects, the subject has a left ventricular ejection fraction of less than about 40%, less than about 41%, less than about 42%, less than about 43%, less than about 44%, less than about 45%, greater than about 46%, less than about 46%, less than about 48%, less than about 49%, or less than about 50%. Preferably, the subject has a left ventricular ejection fraction of less than 50%.

In certain aspects, the present disclosure provides a method of agonizing β₃-adrenergic receptor and antagonizing β₁-adrenergic receptor, β₂-adrenergic receptor, or both β₁-adrenergic receptor and β₂-adrenergic receptor in a subject in need thereof, the method comprising administering 1) a therapeutically effective amount of vibegron to the subject to agonize β₃-adrenergic receptor and 2) a therapeutically effective amount of a therapeutic agent that antagonizes β₁-adrenergic receptor, that antagonizes β₂-adrenergic receptor, or that antagonizes both β₁-adrenergic receptor and β₂-adrenergic receptor. In some aspects, the subject suffers from heart failure. In some aspects, the subject has heart failure with preserved ejection fraction (HFpEF), one or more symptoms thereof, or both. In some aspects, the subject has heart failure with reduced ejection fraction (HFrEF), one or more symptoms thereof, or both. In some aspects, the subject has heart failure with mid-range ejection fraction (HFmrEF), one or more symptoms thereof, or both.

In certain aspects, the present disclosure provides a method of agonizing β₃-adrenergic receptor in a subject in need thereof, the method comprising administering a therapeutically effective amount of vibegron to the subject. In some aspects, the subject suffers from heart failure. In some aspects, the subject has heart failure with preserved ejection fraction (HFpEF), one or more symptoms thereof, or both. In some aspects, the subject has heart failure with reduced ejection fraction (HFrEF), one or more symptoms thereof, or both. In some aspects, the subject has heart failure with mid-range ejection fraction (HFmrEF), one or more symptoms thereof, or both.

In some aspects of the methods described herein, agonizing β₃-adrenergic receptor comprises agonizing β₃-adrenergic receptor in the cardiovascular system of the subject. In some aspects, agonizing β₃-adrenergic receptor comprises agonizing β₃-adrenergic receptor in the myocardium of the subject. In some aspects, the vibegron is administered orally. In some aspects, the vibegron is administered parenterally. In some aspects, the vibegron is administered intravenously. In some aspects, the therapeutically effective amount of vibegron is from about 50 mg to about 450 mg per day. In some aspects, the therapeutically effective amount of vibegron is from about 50 mg to about 300 mg per day. In some aspects, the therapeutically effective amount of vibegron is from about 100 mg to about 200 mg per day. In some aspects, the vibegron is administered once per day. In some aspects, the vibegron is administered with a meal. In some aspects, the vibegron is administered without a meal. In some aspects, the vibegron is administered as a free base. In some aspects, wherein vibegron is administered as a pharmaceutically acceptable salt thereof.

In some aspects of the methods described herein, agonizing β₃-adrenergic receptor further comprises administering to the subject a second therapeutic agent. In some aspects, wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker-neprolysin inhibitor antagonist, an alpha-2 receptor agonist, and a combination thereof.

Typically, and in certain aspects of the methods described herein, the subject can experience one or more beneficial outcomes after administration of vibegron over a treatment period including, but not limited to, the beneficial outcomes described below.

In certain aspects, the present disclosure provides methods of increasing left ventricular ejection fraction (LVEF) as measured by magnetic resonance imaging (MRI), ultrasound, echocardiography, and/or computed tomography (CT) in a subject having or at risk of having heart failure with reduced ejection fraction (HFrEF). In some aspects, the subject having HFrEF experiences an increase in left ventricular ejection fraction (LVEF) as measured by magnetic resonance imaging (MRI), ultrasound, echocardiography, and/or computed tomography (CT) after a period of from about 3 months to about 6 months. In some aspects, the subject experiences an increase in left ventricular ejection fraction (LVEF) as measured by magnetic resonance imaging (MRI), ultrasound, echocardiography, and/or computed tomography (CT) after a period of about 3 months, about 4 months, about 5 months, and/or about 6 months.

In some aspects, the subject experiences a change in hemodynamics as assessed by right heart catheterization after a treatment period. In some aspects, the subject experiences a change in one or more parameters after a treatment period including, but not limited to, cardiac output, pulmonary wedge pressure, pulmonary and systemic vascular resistance, and QT interval, as assessed by right heart catheterization. In some aspects, the subject experiences a change in one or more of these parameters after a treatment period of from about 3 hours to about 6 months. In some aspects, the subject experiences a change in one or more of these parameters after a treatment period of about 3 hours, about 4 hours, about 5 hours, about 6 hours, about 12 hours, about 18 hours, about 1 day, about 3 days, about 5 days, about 1 week, about 2 weeks, about 3 weeks, about 1 month, about 2 months, about 3 months, about 4 months, about 5 months, and/or about 6 months.

Subjects with heart failure can experience pulmonary edema. In some aspects, the subject experiences an improvement in lung function and/or symptoms associated with pulmonary edema. In some aspects, the subject experiences an improvement in lung function and/or symptoms associated with congestive pulmonary edema after a treatment period of from about 3 hours to about 6 months. In some aspects, the subject experiences an improvement in lung function and/or symptoms associated with pulmonary edema after a treatment period of about 3 hours, about 4 hours, about 5 hours, about 6 hours, about 12 hours, about 18 hours, about 1 day, about 3 days, about 5 days, about 1 week, about 2 weeks, about 3 weeks, about 1 month, about 2 months, about 3 months, about 4 months, about 5 months, and/or about 6 months.

In some aspects, the subject experiences one or more of the following parameters after a treatment period: a decrease in N-terminal brain natriuretic peptide (NT proBNP), an increase in 6 minute walking distance; an increase in cardiac output/stroke volume (CO/SV), a reduction in left ventricular internal diameter end diastole (LVIDd), an improvement in diastolic function, a reduction in left atrial (LA) volume, a reduction in LV (left ventricle) diameter, no change in QT interval, and an improvement in NHYA functional class. In some aspects, the subject experiences a change in one or more of these parameters after a treatment period of from about 3 hours to about 12 months. In some aspects, the subject experiences a change in one or more of these parameters after a treatment period of about 3 hours, about 4 hours, about 5 hours, about 6 hours, about 12 hours, about 18 hours, about 1 day, about 3 days, about 5 days, about 1 week, about 2 weeks, about 3 weeks, about 1 month, about 2 months, about 3 months, about 4 months, about 5 months, about 6 months, about 7 months, about 8 months, about 9 months, about 10 months, about 11 months, and/or about 12 months.

In some aspects, the subject experiences beneficial cardiac structural remodeling after a treatment period. In some aspects, the subject experiences beneficial cardiac structural remodeling after a treatment period of about 3 months to about 18 months. In some aspects, the subject experiences beneficial cardiac structural remodeling after a treatment period of about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, and/or about 18 months.

In certain aspects, the subject having or at risk of having HFpEF experiences a change in left ventricular mass index (LVMI) after a treatment period. In some aspects, the subject experiences a change in left ventricular mass index (LVMI) after a treatment period of about 3 months to about 18 months. In some aspects, the subject experiences a change in left ventricular mass index (LVMI) after a treatment period of about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, and/or about 18 months. In certain aspects, the left ventricular mass index is measured in g/m 2 and is defined as left ventricular mass divided by body surface as measured by echocardiography or cardiac MRI.

In certain aspects, the subject having or at risk of having HFpEF experiences one or more parameters selected from a reduction in cardiac fibrosis, a change in diastolic function, and a reduction in left atrial dimension (see, for example, Yamamoto et al., Eur. J. Heart Fail. 2013; 15(1): 110-118) after a treatment period. In some aspects, the subject experiences a change in one or more of these parameters after a treatment period of about 3 months to about 18 months. In some aspects, the subject experiences a change in one or more of these parameters after a after a treatment period of about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, and/or about 18 months.

In some aspects, the subject experiences a shortened duration of hospital stay relative to a subject who has not received vibegron or as compared to the subject's experience before receiving vibegron. In some aspects, the subject experiences an increase in the number of days between hospital stays relative to a subject who has not received vibegron or as compared to the subject's experience before receiving vibegron. In some aspects, the subject experiences a reduced risk of hospitalization relative to a subject who has not received vibegron or as compared to the subject's experience before receiving vibegron. In some aspects, the subject experiences an increase in the time before an initial hospitalization relative to a subject who has not received vibegron or as compared to the subject's experience before receiving vibegron. In some aspects, the subject experiences a fewer number of hospitalizations relative to a subject who has not received vibegron or as compared to the subject's experience before receiving vibegron. In some aspects, the subject experiences a reduced risk of death relative to a subject who has not received vibegron or as compared to the subject's experience before receiving vibegron. In some aspects, the subject experiences one or more of these parameters after a treatment period of from about 3 hours to about 6 months. In some aspects, the subject experiences one or more of these parameters after a treatment period of about 3 hours, about 4 hours, about 5 hours, about 6 hours, about 12 hours, about 18 hours, about 1 day, about 3 days, about 5 days, about 1 week, about 2 weeks, about 3 weeks, about 1 month, about 2 months, about 3 months, about 4 months, about 5 months, and/or about 6 months.

The effective dosage of active ingredient employed to, for example, agonize β₃-adrenergic receptor in the cardiovascular system of the subject or the myocardium of the subject, to treat heart failure and/or one or more symptoms thereof, to treat or prevent heart failure with preserved ejection fraction (HFpEF) or heart failure with reduced ejection fraction (HFrEF), can vary depending on the mode of administration and the severity of the condition. Such dosage may be ascertained readily by a person skilled in the art. In certain aspects, the amount of vibegron administered per day is from about 1 mg to about 1000 mg, from about 2 mg to about 1000 mg, from about 3 mg to about 1000 mg, from about 4 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 40 mg to about 1000 mg, from about 50 mg to about 1000 mg, from about 60 mg to about 1000 mg, from about 70 mg to about 1000 mg, from about 80 mg to about 1000 mg, from about 90 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In certain aspects, the present disclosure provides methods of treating or preventing heart failure and/or one or more symptoms thereof, the method comprising orally administering to a subject in need thereof an amount of from about 1 mg to about 100 mg of vibegron per day. In some aspects, the amount of vibegron administered per day is about 1 mg to about 90 mg, about 1 mg to about 80 mg, about 1 mg to about 70 mg, about 1 mg to about 60 mg, or about 1 mg to about 50 mg.

In certain aspects, the present disclosure provides methods of treating or preventing heart failure with preserved ejection fraction (HFpEF) and/or one or more symptoms thereof, the method comprising orally administering to a subject in need thereof an amount of from about 1 mg to about 100 mg of vibegron per day. In some aspects, the amount of vibegron administered per day is about 1 mg to about 90 mg, about 1 mg to about 80 mg, about 1 mg to about 70 mg, about 1 mg to about 60 mg, or about 1 mg to about 50 mg. In preferred aspects, the subject has HFpEF with an ejection fraction of greater than about 50% and is administered an amount of from about 1 mg to about 100 mg of vibegron per day.

In certain aspects, the present disclosure provides methods of treating or preventing heart failure with reduced ejection fraction (HFrEF) and/or one or more symptoms thereof, the method comprising orally administering to a subject in need thereof an amount of from about 1 mg to about 100 mg of vibegron per day. In some aspects, the amount of vibegron administered per day is about 1 mg to about 90 mg, about 1 mg to about 80 mg, about 1 mg to about 70 mg, about 1 mg to about 60 mg, or about 1 mg to about 50 mg. In preferred aspects, the subject has HFrEF with an ejection fraction of less than about 40% and is administered an amount of from about 1 mg to about 100 mg of vibegron per day.

In certain aspects, the present disclosure provides methods of treating or preventing heart failure and/or one or more symptoms thereof, the method comprising orally administering to a subject in need thereof an amount of from about 5 mg to about 100 mg of vibegron per day. In some aspects, the amount of vibegron administered per day is about 5 mg to about 90 mg, about 5 mg to about 80 mg, about 5 mg to about 70 mg, about 5 mg to about 60 mg, or about 5 mg to about 50 mg.

In certain aspects, the present disclosure provides methods of treating or preventing heart failure with preserved ejection fraction (HFpEF) and/or one or more symptoms thereof, the method comprising orally administering to a subject in need thereof an amount of from about 5 mg to about 100 mg of vibegron per day. In some aspects, the amount of vibegron administered per day is about 5 mg to about 90 mg, about 5 mg to about 80 mg, about 5 mg to about 70 mg, about 5 mg to about 60 mg, or about 5 mg to about 50 mg. In preferred aspects, the subject has HFpEF with an ejection fraction of greater than about 50% and is administered an amount of from about 5 mg to about 100 mg of vibegron per day.

In certain aspects, the present disclosure provides methods of treating or preventing heart failure with reduced ejection fraction (HFrEF)and/or one or more symptoms thereof, the method comprising orally administering to a subject in need thereof an amount of from about 5 mg to about 100 mg of vibegron per day. In some aspects, the amount of vibegron administered per day is about 5 mg to about 90 mg, about 5 mg to about 80 mg, about 5 mg to about 70 mg, about 5 mg to about 60 mg, or about 5 mg to about 50 mg. In preferred aspects, the subject has HFrEF with an ejection fraction of less than about 40% and is administered an amount of from about 5 mg to about 100 mg of vibegron per day.

In certain aspects, the present disclosure provides methods of treating or preventing heart failure and/or one or more symptoms thereof, the method comprising orally administering to a subject in need thereof an amount of from about 45 mg to about 600 mg, from about 45 mg to about 450 mg, or from about 45 mg to about 300 mg of vibegron per day. In some aspects, the method comprises orally administering from about 50 mg to about 600 mg, from about 50 mg to about 450 mg, or preferably from about 50 mg to about 300 mg of vibegron per day.

In certain aspects, the present disclosure provides methods of treating or preventing heart failure with preserved ejection fraction (HFpEF) and/or one or more symptoms thereof, the method comprising orally administering to a subject in need thereof an amount of from about 45 mg to about 600 mg, from about 45 mg to about 450 mg, or from about 45 mg to about 300 mg of vibegron per day. In some aspects, the method comprises orally administering from about 50 mg to about 600 mg, from about 50 mg to about 450 mg, or preferably from about 50 mg to about 300 mg of vibegron per day. In preferred aspects, the subject has HFpEF with an ejection fraction of greater than about 50% and is administered an amount of from about 50 mg to about 300 mg of vibegron per day.

In certain aspects, the present disclosure provides methods of treating or preventing heart failure with reduced ejection fraction (HFrEF) and/or one or more symptoms thereof, the method comprising orally administering to a subject in need thereof an amount of from about 45 mg to about 600 mg, from about 45 mg to about 450 mg, or from about 45 mg to about 300 mg of vibegron per day. In some aspects, the method comprises orally administering from about 50 mg to about 600 mg, from about 50 mg to about 450 mg, or preferably from about 50 mg to about 300 mg of vibegron per day. In preferred aspects, the subject has HFrEF with an ejection fraction of less than about 40% and is administered an amount of from about 50 mg to about 300 mg of vibegron per day.

In certain aspects, the present disclosure provides methods of treating or preventing heart failure and/or one or more symptoms thereof, the method comprising orally administering to a subject in need thereof an amount of from 25 mg to 500 mg of vibegron per day. In some aspects, the amount of vibegron administered per day is about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 150 mg, about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg.

In certain aspects, the present disclosure provides methods of treating or preventing heart failure with preserved ejection fraction (HFpEF) and/or one or more symptoms thereof, the method comprising orally administering to a subject in need thereof an amount of from 25 mg to 500 mg of vibegron per day. In some aspects, the amount of vibegron administered per day is about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 150 mg, about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg.

In certain aspects, the present disclosure provides methods of treating or preventing heart failure with reduced ejection fraction (HFrEF) and/or one or more symptoms thereof, the method comprising orally administering to a subject in need thereof an amount of from 25 mg to 500 mg of vibegron per day. In some aspects, the amount of vibegron administered per day is about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 150 mg, about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg.

In some aspects, the amount of vibegron administered per day in the methods described herein is from about 25 mg to about 600 mg, about 50 mg to about 550 mg, about 75 mg to about 500 mg, about 90 mg to about 450 mg, about 100 mg to about 400 mg, about 125 mg to about 350 mg, or about 150 mg to about 300 mg. In some aspects, the present disclosure provides methods of treating or preventing heart failure and/or one or more symptoms thereof, the method comprising orally administering to a subject in need thereof an amount of from about 100 mg to about 200 mg or from about 200 mg to about 300 mg of vibegron per day.

In some aspects, the amount of vibegron administered per day in the methods described herein is from about 60 mg to about 90 mg, from about 65 mg to about 85 mg, or from about 70 mg to about 80 mg. In some aspects, the amount of vibegron administered per day in the methods described herein is from 60 mg to 90 mg, from 65 mg to 85 mg, or from 70 mg to 80 mg.

In some aspects, the amount of vibegron administered per day in the methods described herein is about 1 mg, about 5 mg, about 10 mg, about 15 mg, about 20 mg, 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, about 250 mg, about 275mg, about 300 mg, about 325 mg, about 350 mg, about 375 mg, about 400 mg, about 425 mg, about 450 mg, about 475 mg, or about 500 mg, or a range between any two preceding values.

In some aspects, the amount of vibegron administered per day in the methods described herein is about 1 mg. In some aspects, the amount of vibegron administered per day in the methods described herein is 1 mg. In some aspects, the amount of vibegron administered per day in the methods described herein is about 25 mg. In some aspects, the amount of vibegron administered per day in the methods described herein is 25 mg. In some aspects, the amount of vibegron administered per day in the methods described herein is about 50 mg. In some aspects, the amount of vibegron administered per day in the methods described herein is 50 mg. In some aspects, the amount of vibegron administered per day in the methods described herein is about 75 mg. In some aspects, the amount of vibegron administered per day in the methods described herein is 75 mg. In some aspects, the amount of vibegron administered per day in the methods described herein is about 100 mg. In some aspects, the amount of vibegron administered per day in the methods described herein is 100 mg. In some aspects, the amount of vibegron administered per day in the methods described herein is about 150 mg. In some aspects, the amount of vibegron administered per day in the methods described herein is 150 mg. In some aspects, the amount of vibegron administered per day in the methods described herein is about 200 mg. In some aspects, the amount of vibegron administered per day in the methods described herein is 200 mg. In some aspects, the amount of vibegron administered per day in the methods described herein is about 250 mg. In some aspects, the amount of vibegron administered per day in the methods described herein is 250 mg. In some aspects, the amount of vibegron administered per day in the methods described herein is about 300 mg. In some aspects, the amount of vibegron administered per day in the methods described herein is 300 mg. In some aspects, the amount of vibegron administered per day in the methods described herein is about 350 mg. In some aspects, the amount of vibegron administered per day in the methods described herein is 350 mg. In some aspects, the amount of vibegron administered per day in the methods described herein is about 400 mg. In some aspects, the amount of vibegron administered per day in the methods described herein is 400 mg. In some aspects, the amount of vibegron administered per day in the methods described herein is about 450 mg. In some aspects, the amount of vibegron administered per day in the methods described herein is 450 mg. In some aspects, the amount of vibegron administered per day in the methods described herein is about 500 mg. In some aspects, the amount of vibegron administered per day is 500 mg.

In some aspects of the methods described herein, a first dose of vibegron is administered for a first period and a second dosage of vibegron is administered thereafter, wherein the second dosage is greater than the first dosage. In some aspects of the methods described herein, the first dosage is from about 1 mg to about 150 mg. In some aspects of the methods described herein, the first dosage is from about 5 mg to about 100 mg. In some aspects of the methods described herein, the first dosage is from about 10 mg to about 75 mg. In some aspects of the methods described herein, the first dosage is from about 25 mg to about 100 mg. In some aspects of the methods described herein, the first dosage is from about 50 mg to about 150 mg. In some aspects of the methods described herein, the first dosage is from about 50 mg to about 100 mg. In some aspects of the methods described herein, the first dosage is from about 50 mg to about 75 mg. In some aspects of the methods described herein, the second dosage is from about 75 mg to about 400 mg. In some aspects of the methods described herein, the second dosage is from about 75 mg to about 350 mg. In some aspects of the methods described herein, the second dosage is from about 75 mg to about 300 mg. In some aspects of the methods described herein, the second dosage is from about 75 mg to about 250 mg. In some aspects of the methods described herein, the second dosage is from about 75 mg to about 200 mg. In some aspects of the methods described herein, the second dosage is from about 75 mg to about 150 mg. In some aspects of the methods described herein, the second dosage is from about 75 mg to about 100 mg. In some aspects of the methods described herein, the first period is from about one week to about 12 weeks. In some aspects of the methods described herein, the first period is from about one week to about 8 weeks. In some aspects of the methods described herein, the first period is from about two weeks to about 12 weeks. In some aspects of the methods described herein, the first period is from about two weeks to about 8 weeks.

In certain aspects of the methods described herein, the dosage of vibegron is from about 0.5 mg/kg/day to about 15 mg/kg/day. In some aspects of the methods described herein, the dosage of vibegron is from about 1 mg/kg/day to about 10 mg/kg/day. In some aspects of the methods described herein, the dosage of vibegron is from about 2 mg/kg/day to about 9 mg/kg/day. In some aspects of the methods described herein, the dosage of vibegron is from about 3 mg/kg/day to about 8 mg/kg/day. In some aspects of the methods described herein, the dosage of vibegron is from about 4 mg/kg/day to about 7 mg/kg/day. In some aspects of the methods described herein, the dosage of vibegron is about 0.5 mg/kg/day, 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 11 mg/kg/day, about 12 mg/kg/day, about 13 mg/kg/day, about 14 mg/kg/day, about 15 mg/kg/day, or a range between any two preceding values.

In some aspects of the methods described herein, vibegron is administered once per day, twice per day, or three times per day. In some aspects of the methods described herein, vibegron is administered once per day.

In some aspects, the method comprises administering vibegron to a subject over a treatment period. The term “treatment period” means the period of time during which the drug is administered to a subject. For example, the treatment period can be from about 2 weeks to about 10 years. In some aspects, the treatment period can be about 2 weeks, about 4 weeks, about 6 weeks, about 8 weeks, about 10 weeks, about 12 weeks, about 14 weeks, about 16 weeks, about 18 weeks, about 20 weeks, about 24 weeks, about 52 weeks, about 74 weeks, about 104 weeks, about 156 weeks, about 208 weeks, about 260 weeks, about 312 weeks, about 364 weeks, about 416 weeks, about 468 weeks, or about 520 weeks. In some aspects, the treatment period can be greater than about 2 weeks, about 4 weeks, about 6 weeks, about 8 weeks, about 10 weeks, about 12 weeks, about 14 weeks, about 16 weeks, about 18 weeks, about 20 weeks, about 24 weeks, about 52 weeks, about 76 weeks, about 104 weeks, about 156 weeks, about 208 weeks, about 260 weeks, about 312 weeks, about 364 weeks, about 416 weeks, about 468 weeks, or about 520 weeks. Other durations of treatment periods and treatment regimens are included throughout the application, and further illustrate, for example, dosage amounts, dosing routes, and dosing intervals that can be used to administer vibegron during the treatment period. The efficacy of the drug can be assessed by measuring certain parameters and calculating the changes from baseline over the treatment period.

In some aspects of the methods described herein, vibegron has an onset of action of about 1 week, about 2 weeks, about 3 weeks, about 4 weeks, about 5 weeks, or about 6 weeks. In some aspects, vibegron has an onset of action of about 1 week. In some aspects, vibegron has an onset of action of about 2 weeks. As shown in the examples, administration of vibegron according to the methods described herein provides onset of action within about 2 weeks of initiation of treatment with vibegron.

In some aspects of the methods described herein, the subject is a mammal. In some aspects the subject is a human or an animal. In some aspects, the subject is a human. In some aspects, the subject is a male. In some aspects, the subject is a female.

In some aspects of the methods described herein, the subject is between the age of about 2 years to about 85 years. In some aspects, the subject is between the age of about 5 years to about 80 years. In some aspects, the subject is between the age of about 10 years to about 75 years. In some aspects, the subject is between the age of about 15 years to about 65 years.

In some aspects of the methods described herein, the subject is over the age of 18 years. In some aspects, the subject is between the age of about 18 years to about 85 years. In some aspects, the subject is between the age of about 18 years to about 80 years. In some aspects, the subject is between the age of about 18 years to about 75 years. In some aspects, the subject is between the age of about 18 years to about 70 years. In some aspects, the subject is between the age of about 18 years to about 65 years.

Any suitable route of administration may be employed for providing a mammal, especially a human with an effective dosage of vibegron in the methods described herein. For example, oral, rectal, topical, parenteral, pulmonary, nasal, and the like may be employed. Dosage forms include tablets, troches, dispersions, suspensions, solutions, capsules, creams, ointments, aerosols, and the like.

In some aspects of the methods described herein, vibegron is administered with food. In some aspects of the methods described herein, vibegron is administered after a meal. In some aspect of the methods described herein, vibegron is administered within 60 minutes after a meal, within 2 hours after a meal, or within 3 hours after a meal.

In some aspects of the methods described herein, vibegron is administered without food. In some aspects of the methods described herein, vibegron is administered before a meal. In some aspects of the methods described herein, vibegron is administered more than three hours before a meal, more than two hours before a meal, or more than 60 minutes before a meal.

In some aspects of the methods described herein, the method comprises crushing a pharmaceutical unit dose composition comprising vibegron before administration to a subject. In some aspects of the methods described herein, the subject is orally administered a crushed pharmaceutical unit dose comprising vibegron.

In some aspects of the methods described herein, the subject is concomitantly receiving, taking, or otherwise being exposed to a cytochrome P450 inhibitor, such as a CYP3A inhibitor, and with drugs that are substrates of the following CYPs: CYP1A2, 2B6, 2C8, 2C9, 2C19, 2D6, and 3A4.

In some aspects of the methods described herein, the subject is concomitantly receiving, taking, or otherwise being exposed to a P-glycoprotein inhibitor.

CYP3A/P-glycoprotein inhibitors include but are not limited to amiodarone, carvedilol, clarithromycin, dronedarone, itraconazole, lapatinib, lopinavir and ritonavir, propafenone, quinidine, ranolazine, ritonavir, saquinavir and ritonavir, telaprevir, tipranavir and ritonavir, verapamil, curcumin, cyclosporine A, eltrombopag, atazanavir and ritonavir, clarithromycin, cyclosporine, erythromycin, gemfibrozil, rifampin (single dose), simeprevir, p-aminohippuric acid (PAH), probenecid, teriflunomide, cimetidine, dolutegravir, isavuconazole, ranolazine, trimethoprim, and vandetanib.

Changes from baseline in blood pressure (BP) and heart rate (HR) for the subjects taking vibegron according to the methods described herein are not substantially different for the subjects taking a placebo. In some aspects, the subject experiences a mean maximum change of systolic blood pressure (SBP) from baseline over a treatment period, and the mean maximum change is less than 2.0 mm Hg, less than 1.9 mm Hg, less than 1.8 mm Hg, less than 1.7 mm Hg, less than 1.6 mm Hg, less than 1.5 mm Hg, less than 1.4 mm Hg, less than 1.3 mm Hg, less than 1.2 mm Hg, less than 1.1 mm Hg, less than 1.0 mm Hg, less than 0.9 mm Hg, less than 0.8 mm Hg, less than 0.7 mm Hg, less than 0.6 mm Hg, or less than 0.5 mm Hg from that of a subject taking a placebo.

In some aspects, of the methods described herein the subject experiences a mean maximum change of diastolic blood pressure (DBP) from baseline over a treatment period, and the mean maximum change is less than 2.0 mm Hg, less than 1.9 mm Hg, less than 1.8 mm Hg, less than 1.7 mm Hg, less than 1.6 mm Hg, less than 1.5 mm Hg, less than 1.4 mm Hg, less than 1.3 mm Hg, less than 1.2 mm Hg, less than 1.1 mm Hg, less than 1.0 mm Hg, less than 0.9 mm Hg, less than 0.8 mm Hg, less than 0.7 mm Hg, less than 0.6 mm Hg, or less than 0.5 mm Hg from that of a subject taking a placebo.

In some aspects of the methods described herein, the subject experiences a mean maximum change of systolic blood pressure (SBP) from baseline over a treatment period of less than 10 mm Hg, less than 9.5 mm Hg, less than 9 mm Hg, less than 8.5 mm Hg, less than 8 mm Hg, less than 7.5 mm Hg, less than 7 mm Hg, less than 6.5 mm Hg, less than 6 mm Hg, less than 5.5 mm Hg, or less than 5 mm Hg.

In some aspects of the methods described herein, the subject experiences a mean maximum change of diastolic blood pressure (DBP) from baseline over a treatment period of less than 7 mm Hg, less than 6.5 mm Hg, less than 6 mm Hg, less than 5.5 mm Hg, less than 5 mm Hg, less than 4.5 mm Hg, less than 4 mm Hg, less than 3.5 mm Hg, less than 3 mm Hg, less than 2.5 mm Hg, or less than 2 mm Hg.

In some aspects of the methods described herein, the subject experiences a smaller maximum mean change in systolic blood pressure than a subject receiving a therapeutically effective amount of mirabegron. In some aspects, the subject receiving vibegron experiences an increase in maximum mean change in systolic blood pressure of from about 1.5 mm Hg to about 4.0 mm Hg smaller than a subject receiving a therapeutically effective amount of mirabegron.

In some aspects of the methods described herein, the subject receiving vibegron experiences a smaller mean 24 hour change in systolic blood pressure than a subject receiving a therapeutically effective amount of mirabegron. In some aspects, the subject receiving vibegron experiences an increase in mean 24 hour change in systolic blood pressure of from about 1.0 mm Hg to about 10.0 mm Hg smaller than a subject receiving a therapeutically effective amount of mirabegron.

In some aspects of the methods described herein, the subject experiences a smaller maximum mean increase in heart rate than a subject receiving a therapeutically effective amount of mirabegron. In some embodiments, the subject receiving vibegron experiences an increase in maximum mean increase in heart rate that is from about 2 bpm to about 14 bpm smaller than a subject receiving a therapeutically effective amount of mirabegron.

In certain aspects of the methods described herein, the subject's contractile function (e.g., in a subject having HFrEF) is increased or improved relative to a subject who has not received vibegron or as compared to the subject's contractile function before receiving vibegron. In certain aspects, the increase or improvement in the subject's contractile function (e.g., in a subject having HFrEF) is measured by fractional shortening. In some aspects the subject's fractional shortening (e.g., in a subject having HFrEF) is increased relative to a subject who has not received vibegron.

In some aspects of the methods described herein, the subject's fractional shortening is increased by about 2% to about 25%. In some aspects, the subject's fractional shortening is increased by about 2% to about 24%. In some aspects, the subject's fractional shortening is increased by about 2% to about 23%. In some aspects, the subject's fractional shortening is increased by about 2% to about 22%. In some aspects, the subject's fractional shortening is increased by about 2% to about 21%. In some aspects, the subject's fractional shortening is increased by about 2% to about 20%. In some aspects, the subject's fractional shortening is increased by about 2% to about 19%. In some aspects, the subject's fractional shortening is increased by about 2% to about 18%. In some aspects, the subject's fractional shortening is increased by about 2% to about 17%. In some aspects, the subject's fractional shortening is increased by about 2% to about 16%. In some aspects, the subject's fractional shortening is increased by about 2% to about 15%. In some aspects, the subject's fractional shortening is increased by about 2% to about 14%. In some aspects, the subject's fractional shortening is increased by about 2% to about 13%. In some aspects, the subject's fractional shortening is increased by about 2% to about 12%. In some aspects, the subject's fractional shortening is increased by about 2% to about 10%. In some aspects, the subject's fractional shortening is increased by about 2% to about 9%. In some aspects, the subject's fractional shortening is increased by about 2% to about 8%. In some aspects, the subject's fractional shortening is increased by about 2% to about 7%. In some aspects, the subject's fractional shortening is increased by about 2% to about 6%. In some aspects, the subject's fractional shortening is increased by about 2% to about 5%.

In some aspects of the methods described herein, the subject's fractional shortening is increased by about 3% to about 15%. In some aspects, the subject's fractional shortening is increased by about 4% to about 14%. In some aspects, the subject's fractional shortening is increased by about 5% to about 13%. In some aspects, the subject's fractional shortening is increased by about 6% to about 12%. In some aspects, the subject's fractional shortening is increased by about 7% to about 11%. In some aspects, the subject's fractional shortening is increased by about 7% to about 10%.

In some aspects of the methods described herein, the subject's fractional shortening is increased by at least about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50%, or by an interval between any of the preceding values.

In certain aspects of the methods described herein, the increase or improvement in the subject's contractile function (e.g., in a subject having HFrEF) is measured by ejection fraction. In some aspects, the subject's ejection fraction (e.g., in a subject having HFrEF) is increased relative to a subject who has not received vibegron.

In some aspects of the methods described herein, the subject's ejection fraction is increased by about 2% to about 25% more than a subject who has not received vibegron. In some aspects, the subject's ejection fraction is increased by about 2% to about 24%. In some aspects, the subject's ejection fraction is increased by about 2% to about 23%. In some aspects, the subject's ejection fraction is increased by about 2% to about 22%. In some aspects, the subject's ejection fraction is increased by about 2% to about 21%. In some aspects, the subject's ejection fraction is increased by about 2% to about 20%. In some aspects, the subject's ejection fraction is increased by about 2% to about 19%. In some aspects, the subject's ejection fraction is increased by about 2% to about 18%. In some aspects, the subject's ejection fraction is increased by about 2% to about 17%. In some aspects, the subject's ejection fraction is increased by about 2% to about 15%. In some aspects, the subject's ejection fraction is increased by about 2% to about 14%. In some aspects, the subject's ejection fraction is increased by about 2% to about 13%. In some aspects, the subject's ejection fraction is increased by about 2% to about 12%. In some aspects, the subject's ejection fraction is increased by about 2% to about 11%. In some aspects, the subject's ejection fraction is increased by about 2% to about 10%. In some aspects, the subject's ejection fraction is increased by about 2% to about 9%. In some aspects, the subject's ejection fraction is increased by about 2% to about 8%. In some aspects, the subject's ejection fraction is increased by about 2% to about 7%. In some aspects, the subject's ejection fraction is increased by about 2% to about 6%. In some aspects, the subject's ejection fraction is increased by about 2% to about 5%.

In some aspects of the methods described herein, the subject's ejection fraction is increased by about 5% to about 25%. In some aspects, the subject's ejection fraction is increased by about 6% to about 24%. In some aspects, the subject's ejection fraction is increased by about 7% to about 23%. In some aspects, the subject's ejection fraction is increased by about 8% to about 22%. In some aspects, the subject's ejection fraction is increased by about 9% to about 21%. In some aspects, the subject's ejection fraction is increased by about 10% to about 20%. In some aspects, the subject's ejection fraction is increased by about 11% to about 19%. In some aspects, the subject's ejection fraction is increased by about 12% to about 18%. In some aspects, the subject's ejection fraction is increased by about 13% to about 17%.

In some aspects of the methods described herein, the subject's ejection fraction is increased by at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, or about 50%, or by an interval between any of the preceding values.

In certain aspects of the methods described herein, the blood volume in the subject's left ventricle (e.g., in a subject having HFrEF) is increased or improved relative to a subject who has not received vibegron or as compared to the subject's blood volume before receiving vibegron. In certain aspects, the increase or improvement in the subject's blood volume (e.g., in a subject having HFrEF) is measured by stroke volume in the subject's left ventricle. In some aspects, stroke volume in the subject's left ventricle (e.g., in a subject having HFrEF) is increased relative to a subject who has not received vibegron.

In some aspects of the methods described herein, the stroke volume in the subject's left ventricle is increased by about 2 μL to about 25 μL relative to a subject who has not received vibegron. In some aspects, the stroke volume is increased by about 2 μL to about 24 μL. In some aspects, the stroke volume is increased by about 2 μL to about 23 μL. In some aspects, the stroke volume is increased by about 2 μL to about 22 μL. In some aspects, the stroke volume is increased by about 2 μL to about 21 μL. In some aspects, the stroke volume is increased by about 2 μL to about 20 μL. In some aspects, the stroke volume is increased by about 2 μL to about 19 μL. In some aspects, the stroke volume is increased by about 2 μL to about 18 μL. In some aspects, the stroke volume is increased by about 2 μL to about 17 μL In some aspects, the stroke volume is increased by about 2 μL to about 16 μL. In some aspects, the stroke volume is increased by about 2 μL to about 15 μL. In some aspects, the stroke volume is increased by about 2 μL to about 14 μL In some aspects, the stroke volume is increased by about 2 μL to about 13 μL. In some aspects, the stroke volume is increased by about 2 μL to about 12 μL. In some aspects, the stroke volume is increased by about 2 μL to about 11 μL. In some aspects, the stroke volume is increased by about 2 μL to about 10 μL. In some aspects, the stroke volume is increased by about 2 μL to about 9 μL. In some aspects, the stroke volume is increased by about 2 μL to about 8 μL. In some aspects, the stroke volume is increased by about 2 μL to about 5 μL.

In some aspects of the methods described herein, the stroke volume is increased by about 10 μL to about 25 μL. In some aspects, the stroke volume is increased by about 11 μL to about 24 μL. In some aspects, the stroke volume is increased by about 12 μL to about 23 μL. In some aspects, the stroke volume is increased by about 13 μL to about 22 μL. In some aspects, the stroke volume is increased by about 14 μL to about 21 μL. In some aspects, the stroke volume is increased by about 15 μL to about 20 μL. In some aspects, the stroke volume in the subject's left ventricle is increased by at least about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 25 μL, about 30 μL, about 35 μL, about 40 μL, about 45 μL, or about 50 μL, or by an interval between any of the preceding values.

In some aspects of the methods described herein, the stroke volume in the subject's left ventricle is increased by about 1% to about 100% relative to a subject who has not received vibegron. In some aspects, the stroke volume is increased by about 5% to about 95% relative to a subject who has not received vibegron. In some aspects, the stroke volume is increased by about 10% to about 90% relative to a subject who has not received vibegron. In some aspects, the stroke volume is increased by about 15% to about 85% relative to a subject who has not received vibegron. In some aspects, the stroke volume is increased by about 20% to about 80% relative to a subject who has not received vibegron. In some aspects, the stroke volume is increased by about 25% to about 75% relative to a subject who has not received vibegron. In some aspects, the stroke volume is increased by about 30% to about 70% relative to a subject who has not received vibegron. In some aspects, the stroke volume is increased by about 30% to about 65% relative to a subject who has not received vibegron. In some aspects, the stroke volume is increased by about 35% to about 65% relative to a subject who has not received vibegron. In some aspects, the stroke volume is increased by about 40% to about 60% relative to a subject who has not received vibegron.

In some aspects of the methods described herein, the stroke volume is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 50%, about 55%, about 60%,about 65%, about 70%, about 75%, about 80%, about 90%, about 95%, or about 100% relative to a subject who has not received vibegron, or by an interval between any of the preceding values.

In certain aspects of the methods described herein, the increase or improvement in the subject's blood volume (e.g., in a subject having HFrEF) is measured by cardiac output. In some aspects, the subject's cardiac output (e.g., in a subject having HFrEF) is increased relative to a subject who has not received vibegron.

In some aspects of the methods described herein, the subject's cardiac output is increased by about 2 mL/min to about 20 mL/min relative to a subject who has not received vibegron. In some aspects, the subject's cardiac output is increased by about 2 mL/min to about 19 mL/min. In some aspects, the subject's cardiac output is increased by about 2 mL/min to about 18 mL/min. In some aspects, the subject's cardiac output is increased by about 2 mL/min to about 17 mL/min. In some aspects, the subject's cardiac output is increased by about 2 mL/min to about 16 mL/min. In some aspects, the subject's cardiac output is increased by about 2 mL/min to about 15 mL/min. In some aspects, the subject's cardiac output is increased by about 2 mL/min to about 14 mL/min. In some aspects, the subject's cardiac output is increased by about 2 mL/min to about 14 mL/min. In some aspects, the subject's cardiac output is increased by about 2 mL/min to about 13 mL/min. In some aspects, the subject's cardiac output is increased by about 2 mL/min to about 12 mL/min. In some aspects, the subject's cardiac output is increased by about 2 mL/min to about 11 mL/min. In some aspects, the subject's cardiac output is increased by about 2 mL/min to about 10 mL/min. In some aspects, the subject's cardiac output is increased by about 2 mL/min to about 9 mL/min. In some aspects, the subject's cardiac output is increased by about 2 mL/min to about 8 mL/min. In some aspects, the subject's cardiac output is increased by about 2 mL/min to about 7 mL/min. In some aspects, the subject's cardiac output is increased by about 2 mL/min to about 6 mL/min. In some aspects, the subject's cardiac output is increased by about 2 mL/min to about 5 mL/min.

In some aspects of the methods described herein, the subject's cardiac output is increased by about 2 mL/min to about 15 mL/min. In some aspects, the subject's cardiac output is increased by about 3 mL/min to about 14 mL/min. In some aspects, the subject's cardiac output is increased by about 4 mL/min to about 13 mL/min. In some aspects, the subject's cardiac output is increased by about 5 mL/min to about 12 mL/min. In some aspects, the subject's cardiac output is increased by about 6 mL/min to about 11 mL/min. In some aspects, the subject's cardiac output is increased by about 7 mL/min to about 10 mL/min.

In some aspects of the methods described herein, the subject's cardiac output is increased by at least about 2 mL/min, about 3 mL/min, about 4 mL/min, about 5 mL/min, about 6 mL/min, about 7 mL/min, about 8 mL/min, about 9 mL/min, about 10 mL/min, about 11 mL/min, about 12 mL/min, about 13 mL/min, about 14 mL/min, about 15 mL/min, about 20 mL/min, about 25 mL/min, about 30 mL/min, about 35 mL/min, about 40 mL/min, about 45 mL/min, or about 50 mL/min, or by an interval between any of the preceding values.

In some aspects of the methods described herein, the cardiac output in the subject's left ventricle is increased by about 1% to about 100% relative to a subject who has not received vibegron. In some aspects, the cardiac output is increased by about 5% to about 95% relative to a subject who has not received vibegron. In some aspects, the cardiac output is increased by about 10% to about 90% relative to a subject who has not received vibegron. In some aspects, cardiac output is increased by about 15% to about 85% relative to a subject who has not received vibegron. In some aspects, the cardiac output is increased by about 20% to about 80% relative to a subject who has not received vibegron. In some aspects, the cardiac output is increased by about 25% to about 75% relative to a subject who has not received vibegron. In some aspects, the cardiac output is increased by about 30% to about 70% relative to a subject who has not received vibegron. In some aspects, the cardiac output is increased by about 30% to about 65% relative to a subject who has not received vibegron. In some aspects, the cardiac output is increased by about 35% to about 65% relative to a subject who has not received vibegron. In some aspects, the cardiac output is increased by about 40% to about 60% relative to a subject who has not received vibegron.

In some aspects of the methods described herein, the cardiac output is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 50%, about 55%, about 60%, about 65%, about 70%, about 75%, about 80%, about 90%, about 95%, or about 100% relative to a subject who has not received vibegron, or by an interval between any of the preceding values.

In certain aspects, the methods of the present disclosure may further comprise administering to the subject a second therapeutic agent. In certain aspects, the second therapeutic agent can be an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker-neprolysin inhibitor antagonist (also referred to as “ARNI”), an alpha-2 receptor agonist, or a combination thereof. In some aspects, the antihyperlipoproteinemic agent is selected from HMG-CoA reductase inhibitors such as atorvastatin, fluvastatin, lovastatin, pravastatin, rosuvastatin, or simvastatin, or other agents such as ezetimibe, fibric acid derivatives, niacin, or bile acid sequestrants, such as cholestyramine, colestipol, or colesevelam. In some aspects, the blood coagulant is selected from aprotinin, tranexamic acid, epsilon-aminocaproic acid, or aminomethylbenzoic acid. In some aspects, the antiarrhythmic agent is selected from amiodarone, flecainide, ibutilide, lidocaine, procainamide, propafenone, quinidine, or tocainide. In some aspects, the antihypertensive agent is selected from furosemide, bumetanide, amiloridhydralazine, minoxidil, spironolactone, clonidine, or methyldopa. In some aspects, the vasopressor is selected from norepinephrine, epinephrine, vasopressin, dopamine, phenylephrine, or dobutamine. In some aspects, the diuretic is selected from chlorthalidone, hydrochorothiazide, metolazone, indapamide, bumetanide, ethacrynic acid, or furosemide. In some aspects, the statin is selected from atorvastatin, fluvastatin, lovastatin, pravastatin, rosuvastatin, or simvastatin. In some aspects, the anti-platelet agent is selected from aspirin, clopidogrel, ticlopidine, ticagrelor, prasugrel, or cangrelor. In some aspects, the SGLT2 inhibitor is selected from canagliflozin, dapagliflozin, orempagliflozin. In some aspects, the antianginal agent is selected from a nitrate such as isosorbide dinitrate, isosorbide mononitrate, or nitroglycerin; a calcium antagonist such as diltiazem, nifedipine, nimodipine, or verapamil; a beta blocker such as atenolol, pindolol, propranolol, or metoprolol, or ranolazine. In some aspects, the renin-angiotensin system (RAS) blocker is selected from captopril, imidapril, zofenopril, candesartan, delapril, telmisartan, aliskiren, or moexipril. In some aspects, the mineralocorticoid receptor antagonist (MRA) is selected from spironolactone or eplerenone. In some aspects, the ACE inhibitor is selected from benazepril, captopril, enalapril, fosinopril, lisinopril, moexipril, perindopril, or quinapril. In some aspects, the angiotensin receptor blocker is selected from azilsartan, candesartan, eprosartan, irbesartan, losartan, olmesartan, telmisartan, or valsartan. In some aspects, the neprolysin inhibitor antagonist is selected from sacubitril/valsartan. In some aspects, the alpha-2 receptor agonist is selected from guanabenz, guanfacine, clonidine, tizanidine, medetomidine, or dexmedetomidine. Additional second therapeutic agents for use in the methods described herein are described, e.g., in the Physician's Desk Reference, which), which is incorporated herein by reference in its entirety and is updated annually and identifies, e.g., agents described above.

In some aspects, the methods of the present disclosure for treating HFrEF further comprise administering one or more of the following second therapeutic agents: a diuretic, a RAS blocker, a beta blocker, and/or an MRA, statins, anti-platelet therapy, SGLT2 inhibitors, or a combination thereof.

In some aspects, the methods of the present disclosure for treating HFrEF further comprise administering one or more of the following second therapeutic agents: an angiotensin receptor blocker-neprolysin inhibitor antagonist (also referred to as “ARNI”), a beta blocker, an MRA, an SGLT2 inhibitor, or a combination thereof. In some aspects, the methods of the present disclosure for treating HFrEF further comprise administering each of an ARNI, a beta blocker, an MRA, and an SGLT2 inhibitor.

In some aspects, the methods of the present disclosure for treating HFrEF further comprise administering one or more of the following second therapeutic agents: an angiotensin receptor blocker-neprolysin inhibitor antagonist (also referred to as “ARNI”), a beta blocker, an MRA, an SGLT2 inhibitor, an ACE inhibitor, or a combination thereof. In some aspects, the methods of the present disclosure for treating HFrEF further comprise administering each of an ARNI, a beta blocker, an MRA, an SGLT2 inhibitor, and an ACE inhibitor.

In some aspects, the methods of the present disclosure for treating HFpEF further comprise administering one or more of the following second therapeutic agents: a diuretic, a RAS blocker, an SGLT2 inhibitor, an MRA, an antihypertensive agent, or a combination thereof.

In some aspects, the methods of the present disclosure for treating HFpEF further comprise administering one or more of the following second therapeutic agents: one or more diuretics, an antihypertensive agent, an SGLT2 inhibitor, or a combination thereof. In some aspects, the methods of the present disclosure for treating HFpEF further comprise administering each of one or more diuretics, an antihypertensive agent, and an SGLT2 inhibitor. In some aspects, the one or more diuretics comprises Lasix.

### Pharmaceutical Unit Dose Composition

In some aspects, the present disclosure provides compositions including vibegron. In certain aspects, the compositions can include vibegron and be in a form suitable for oral (including through a nasogastric tube), intravesical, rectal, topical, parenteral (including subcutaneous, intramuscular, and intravenous), pulmonary (nasal or buccal inhalation), or nasal administration. The most suitable route in any given case will depend on the nature and severity of the conditions being treated. Compositions may be conveniently presented in unit dosage form and prepared by any of the methods well-known in the art of pharmacy.

In certain aspects, the present disclosure provides pharmaceutical unit dose compositions comprising a dosage of vibegron disclosed herein, wherein the unit dosage composition is suitable for oral administration. Oral dosage forms are recognized by those skilled in the art to include, for example, such forms as liquid formulations, tablets, capsules, and gelcaps. In some aspects, the unit dose compositions are solid dosage forms, such as tablets and capsules. In some aspects, the unit dose compositions are tablets.

Pharmaceutically acceptable excipients are excipients generally recognized as safe such as lactose, microcrystalline cellulose, starch, calcium carbonate, magnesium stearate, stearic acid, talc, colloidal silicon dioxide, mannitol, croscarmellose sodium, hydroxypropyl cellulose. In some aspects, the pharmaceutical unit dose composition disclosed herein comprises a diluent, a disintegrant, a binder, and a lubricant. See generally, Remington's Pharmaceutical Sciences, 20th ed., Mack Publishing, Easton PA (2000), which is incorporated herein by reference in its entirety.

In some aspects, the pharmaceutical unit dose composition disclosed herein comprises mannitol, microcrystalline cellulose, croscarmellose sodium, hydroxypropyl cellulose, and magnesium stearate.

Oral dosage forms can be prepared by standard pharmaceutical manufacturing techniques. Such techniques include, for example, wet granulation, wet milling, fluid bed drying, dry milling, lubrication, tableting, and aqueous film coating.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

In some aspects, the compositions described herein can be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

In some aspects, the pharmaceutical forms suitable for injectable use include sterile aqueous solutions or dispersions and sterile powders for the extemporaneous preparation of sterile inject-able solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

For topical administration to the epidermis, vibegron may be formulated as an ointment, cream, lotion, or as a transdermal patch. Ointments and creams may, for example, be formulated with an aqueous or oily base with the addition of suitable thickening and/or gelling agents.

Formulations suitable for topical administration in the mouth include lozenges comprising vibegron in a flavored base, usually sucrose and acacia or tragacanth; pastilles comprising vibegron in an inert base such as gelatin and glycerin or sucrose and acacia; and mouthwashes comprising vibegron in a suitable liquid carrier.

Solutions or suspensions are applied directly to the nasal cavity by conventional means. In the case of a spray, this may be achieved for example by means of a metering atomizing spray pump.

Administration to the respiratory tract may also be achieved by means of an aerosol formulation in which vibegron is provided in a pressurized pack with a suitable propellant. If compositions are administered as aerosols, for example as nasal aerosols or by inhalation, this can be carried out, for example, using a spray, a nebulizer, a pump nebulizer, an inhalation apparatus, a metered inhaler, or a dry powder inhaler.

In certain aspects, vibegron may be used in combination with other drugs that are used in the treatment/prevention/suppression or amelioration of heart failure described herein. Such other drugs may be administered, by a route and in an amount commonly used therefor, contemporaneously or sequentially with vibegron. When vibegron is used contemporaneously with one or more other drugs, a pharmaceutical unit dosage form containing such other drugs is within the scope of the disclosure. Accordingly, the pharmaceutical compositions of the present disclosure include those that also contain one or more other active ingredients as discussed above, in addition to vibegron.

In some aspects, the pharmaceutical unit dose compositions of the present disclosure comprise from about 1 mg to about 1000 mg of vibegron. In some aspects, the pharmaceutical unit dose compositions of the present disclosure comprise from about 2 mg to about 1000 mg, from about 3 mg to about 1000 mg, from about 4 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 40 mg to about 1000 mg, from about 50 mg to about 1000 mg, from about 60 mg to about 1000 mg, from about 70 mg to about 1000 mg, from about 80 mg to about 1000 mg, from about 90 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg of vibegron.

In some aspects, the pharmaceutical unit dose compositions of the present disclosure comprise from 25 mg to 500 mg of vibegron. In some aspects, the pharmaceutical unit dose compositions of the present disclosure comprise from about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 150 mg, about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg of vibegron.

In some aspects, the pharmaceutical unit dose compositions of the present disclosure comprise from about 55 mg to about 100 mg, from about 60 mg to about 100 mg, from about 65 mg to about 100 mg, from about 70 mg to about 100 mg, from about 75 mg to about 100 mg, from about 80 mg to about 100 mg, from about 85 mg to about 100 mg, from about 90 mg to about 100 mg, or from about 95 mg to about 100 mg of vibegron.

In some aspects, the pharmaceutical unit dose compositions of the present disclosure comprise from about 60 mg to about 90 mg, from about 65 mg to about 85 mg, or from about 70 mg to about 80 mg of vibegron. In some aspects, the pharmaceutical unit dose compositions of the present disclosure comprise from 50 mg to 100 mg, from 60 mg to 90 mg, from 65 mg to 85 mg, or from 70 mg to 80 mg of vibegron.

In some aspects, the pharmaceutical unit dose compositions of the present disclosure comprise from about 45 mg to about 600 mg, from about 45 mg to about 450 mg, or from about 45 mg to about 300 mg of vibegron. In some aspects, the pharmaceutical unit dose compositions of the present disclosure comprise from about 50 mg to about 600 mg, from about 50 mg to about 450 mg, or from about 50 mg to about 300 mg of vibegron.

In some aspects, the pharmaceutical unit dose compositions of the present disclosure comprise about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 185 mg, about 190 mg, about 195 mg, about 200 mg, about 225 mg, about 250 mg, about 275 mg, about 300 mg, about 325 mg, about 350 mg, about 375 mg, about 400 mg, about 450 mg, or about 500 mg of vibegron. In some aspects, the pharmaceutical unit dose compositions of the present disclosure comprise about 50 mg of vibegron. In some aspects, the pharmaceutical unit dose compositions of the present disclosure comprise 50 mg of vibegron. In some aspects, the pharmaceutical unit dose compositions of the present disclosure comprise about 75 mg of vibegron. In some aspects, the pharmaceutical unit dose compositions of the present disclosure comprise 75 mg of vibegron. In some aspects, the pharmaceutical unit dose compositions of the present disclosure comprise about 100 mg of vibegron. In some aspects, the pharmaceutical unit dose compositions of the present disclosure comprise 100 mg of vibegron.

In some aspects, the present disclosure provides pharmaceutical unit dose compositions comprising a dosage of vibegron disclosed herein, wherein the composition is included in, without limitation, a container, pack, dispenser, or combination thereof, together with instructions for administration.

The present disclosure also provides
(1) Vibegron for use in treating or preventing heart failure with preserved ejection fraction (HFpEF), one or more symptoms thereof, or both in a subject in need thereof, wherein a therapeutically effective amount of vibegron is to be administered to the subject.
(2) Vibegron for use according to (1), wherein the subject has a left ventricular ejection fraction of greater than about 40%.
(3) Vibegron for use according to (1) or (2), wherein the subject has a left ventricular ejection fraction of greater than about 50%.
(4) Vibegron for use according to any one of (1) to (3), wherein the therapeutically effective amount of vibegron is from about 90 mg to about 450 mg per day.
(5) Vibegron for use according to (4), wherein the therapeutically effective amount of vibegron is from about 100 mg to about 400 mg per day.
(6) Vibegron for use according (5), wherein the therapeutically effective amount of vibegron is from about 100 mg to about 200 mg per day.
(7) Vibegron for use according to any one of (1) to (6), wherein a second therapeutic agent is to be administered to the subject.
(8) Vibegron for use according to (7), wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, a calcium channel blocker, an alpha-2 receptor agonist, and a combination thereof.
(9) Vibegron for use according to any one of (1) to (8), wherein the vibegron is to be administered orally.
(10) Vibegron for use according to any one of (1) to (9), wherein the vibegron is to be administered intravenously.
(11) Vibegron for use according to any one of (1) to (10), wherein the subject receiving vibegron experiences a smaller maximum mean change in systolic blood pressure than a subject receiving a therapeutically effective amount of mirabegron.
(12) Vibegron for use according to any one of (1) to (11), wherein the subject receiving vibegron experiences an increase in maximum mean change in systolic blood pressure of from about 1.5 mm Hg to about 4.0 mm Hg smaller than a subject receiving a therapeutically effective amount of mirabegron.
(13) Vibegron for use according to any one of (1) to (12), wherein the subject receiving vibegron experiences a smaller mean 24 hour change in systolic blood pressure than a subject receiving a therapeutically effective amount of mirabegron.
(14) Vibegron for use according to any one of (1) to (13), wherein the subject receiving vibegron experiences an increase in mean 24 hour change in systolic blood pressure of from about 1.0 mm Hg to about 10.0 mm Hg smaller than a subject receiving a therapeutically effective amount of mirabegron.
(15) Vibegron for use according to any one of (1) to (14), wherein the subject receiving vibegron experiences a smaller maximum increase in heart rate than a subject receiving a therapeutically effective amount of mirabegron.
(16) Vibegron for use according to any one of (1) to (15), wherein the subject receiving vibegron experiences an increase in maximum mean increase in heart rate that is from about 2 bpm to about 14 bpm smaller than a subject receiving a therapeutically effective amount of mirabegron.
(17) Vibegron for use according to any one of (11) to (16), wherein the therapeutically effective amount of mirabegron is from about 50 mg to about 300 mg.
(18) Vibegron for use according to any one of (1) to (17), wherein treating comprises halting the progression of HFpEF, relieving one or more symptoms associated with HFpEF, or both.
(19) Vibegron for use in treating or preventing heart failure with reduced ejection fraction (HFrEF), one or more symptoms thereof, or both, in a subject in need thereof, wherein a therapeutically effective amount of vibegron is to be administered to the subject.
(20) Vibegron for use according to (19), wherein the subject has a left ventricular ejection fraction less than about 50%.
(21) Vibegron for use according to (19) or (20), wherein the subject has a left ventricular ejection fraction of less than about 40%.
(22) Vibegron for use according to any one of (19) to (21), wherein the therapeutically effective amount of vibegron is from about 90 mg to about 450 mg.
(23) Vibegron for use according to (22), wherein the therapeutically effective amount of vibegron is from about 100 mg to about 400 mg per day.
(24) Vibegron for use according to (23), wherein the therapeutically effective amount of vibegron is from about 100 mg to about 200 mg per day.
(25) Vibegron for use according to any one of (19) to (24), wherein a second therapeutic agent is to be administered to the subject.
(26) Vibegron for use according to (25), wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, a calcium channel blocker, an alpha-2 receptor agonist, and a combination thereof.
(27) Vibegron for use according to any one of (19) to (26), wherein the vibegron is to be administered orally.
(28) Vibegron for use according to any one of (19) to (26), wherein the vibegron is to be administered intravenously.
(29) Vibegron for use according to any one of (19) to (28), wherein the subject receiving vibegron experiences a smaller maximum mean change in systolic blood pressure than a subject receiving a therapeutically effective amount of mirabegron.
(30) Vibegron for use according to any one of (19) to (29), wherein the subject receiving vibegron experiences an increase in maximum mean change in systolic blood pressure of from about 1.5 mm Hg to about 4.0 mm Hg smaller than a subject receiving a therapeutically effective amount of mirabegron.
(31) Vibegron for use according to any one of (19) to (30), wherein the subject receiving vibegron experiences a smaller mean 24 hour change in systolic blood pressure than a subject receiving a therapeutically effective amount of mirabegron.
(32) Vibegron for use according to any one of (19) to (31), wherein the subject receiving vibegron experiences an increase in mean 24 hour change in systolic blood pressure of from about 1.0 mm Hg to about 10.0 mm Hg smaller than a subject receiving a therapeutically effective amount of mirabegron.
(33) Vibegron for use according to any one of (19) to (32), wherein the subject receiving vibegron experiences a smaller maximum increase in heart rate than a subject receiving a therapeutically effective amount of mirabegron.
(34) Vibegron for use according to any one of (19) to (33), wherein the subject receiving vibegron experiences an increase in maximum mean increase in heart rate that is from about 2 bpm to about 14 bpm smaller than a subject receiving a therapeutically effective amount of mirabegron.
(35) Vibegron for use according to any one of (30) to (34), wherein the therapeutically effective amount of mirabegron is from about 50 mg to about 300 mg.
(36) Vibegron for use according to any one of (19) to (35), wherein treating comprises halting the progression of HFrEF, relieving one or more symptoms associated with HFrEF, or both.
(37) Vibegron for use according to any one of (1) to (36), wherein vibegron is to be administered once per day.
(38) Vibegron for use according to (37), wherein vibegron is to be administered with a meal.
(39) Vibegron for use according to (38), wherein vibegron is to be administered without a meal.
(40) Vibegron for use according to any one of (1) to (39), wherein vibegron is to be administered as a free base.
(41) Vibegron for use according to any one of (1) to (39), wherein vibegron is to be administered as a pharmaceutically acceptable salt thereof.
(42) Vibegron for use according to any one of (1) to (41), wherein the subject's contractile function is increased or improved relative to a subject who has not received vibegron or as compared to the subject's contractile function before receiving vibegron.
(43) Vibegron for use according to (42), wherein the increase or improvement in the subject's contractile function is measured by fractional shortening.
(44) Vibegron for use according to (43), wherein the subject's fractional shortening is increased relative to a subject who has not received vibegron.
(45) Vibegron for use according (44), wherein the subject's fractional shortening is increased by about 2% to about 15% more than a subject who has not received vibegron.
(46) Vibegron for use according to (45), wherein the subject's fractional shortening is increased by about 5% to about 10% more than a subject who has not received vibegron.
(47) Vibegron for use according to (42), wherein the increase or improvement in the subject's contractile function is measured by ejection fraction.
(48) Vibegron for use according to (47), wherein the subject's ejection fraction is increased relative to a subject who has not received vibegron.
(49) Vibegron for use according to (48), wherein the subject's ejection fraction is increased by about 2% to about 20% more than a subject who has not received vibegron.
(50) Vibegron for use according to (49), wherein the subject's ejection fraction is increased by about 10% to about 20% more than a subject who has not received vibegron
(51) Vibegron for use according to any one of (1) to (50), wherein blood volume in the subject's left ventricle is increased or improved relative to a subject who has not received vibegron or as compared to the subject's contractile function before receiving vibegron.
(52) Vibegron for use according to (51), wherein the increase or improvement in the subject's blood volume is measured by stroke volume in the subject's left ventricle.
(53) Vibegron for use according to (52), wherein stroke volume in the subject's left ventricle is increased relative to a subject who has not received vibegron.
(54) Vibegron for use according to (53), wherein the stroke volume in the subject's left ventricle is increased by about 25% to about 75% relative to a subject who has not received vibegron.
(55) Vibegron for use according to (54), wherein the stroke volume in the subject's left ventricle is increased by about 30% to about 65% relative to a subject who has not received vibegron.
(56) Vibegron for use according to (49), wherein the increase or improvement in the subject's blood volume is measured by cardiac output.
(57) Vibegron for use according to (56), wherein the subject's cardiac output is increased relative to a subject who has not received vibegron.
(58) Vibegron for use according to (57), wherein the subject's cardiac output is increased by about 25% to about 75% relative to a subject who has not received vibegron.
(59) Vibegron for use according to (58), wherein the subject's cardiac output is increased by about 30% to about 65% relative to a subject who has not received vibegron.
(60) Vibegron for use according to any one of (1) to (59), wherein vibegron has an onset of action of about 4 weeks, about 3 weeks, or about 2 weeks.
(61) Vibegron for use according to (60), wherein vibegron has an onset of action within about 2 weeks.
(63) Vibegron for use according to any one of (1) to (61), wherein the subject experiences one or more of: a shortened duration of hospital stay, an increase in the number of days between hospital stays, a reduced risk of hospitalization, an increase in the time before an initial hospitalization, or a fewer number of hospitalizations relative to a subject who has not received vibegron or as compared to the subject's experience before receiving vibegron.
(63) Vibegron for use in agonizing β₃-adrenergic receptor in a subject in need thereof, wherein a therapeutically effective amount of vibegron is to be administered to the subject in combination with a therapeutically effective amount of a therapeutic agent that antagonizes β₁-adrenergic receptor, that antagonizes β₂-adrenergic receptor, or that antagonizes both β₁-adrenergic receptor and β₂-adrenergic receptor.
(64) A therapeutic agent for use in antagonizing β₁-adrenergic receptor, antagonizing β₂-adrenergic receptor, or antagonizing both β₁-adrenergic receptor and β₂-adrenergic receptor in a subject in need thereof, wherein a therapeutically effective amount of the therapeutic agent is to be administered to the subject in combination with a therapeutically effective amount of vibegron.
(65) Vibegron for use in agonizing β₃-adrenergic receptor and a therapeutic agent for use in antagonizing β₁-adrenergic receptor, β₂-adrenergic receptor, or both β₁-adrenergic receptor and β₂-adrenergic receptor in a subject in need thereof, wherein a combination of a therapeutically effective amount of vibegron and a therapeutically effective amount of the therapeutic agent is to be administered to the subject.
(66) Vibegron for use in agonizing β₃-adrenergic receptor in a subject in need thereof, wherein a therapeutically effective amount of vibegron is to be administered to the subject.
(67) Vibegron for use according to (63) or (66), wherein the subject suffers from heart failure, one or more symptoms thereof, or both.
(68) Vibegron for use according to any one of (63) to (67), wherein the subject has heart failure with preserved ejection fraction (HFpEF), one or more symptoms thereof, or both.
(69) Vibegron for use according to any one of (63) to (67), wherein the subject has heart failure with reduced ejection fraction (HFrEF), one or more symptoms thereof, or both.
(70) Vibegron for use according to any one of (63) to (67), wherein the subject has heart failure with mid-range ejection fraction (HFmrEF), one or more symptoms thereof, or both.
(71) Vibegron for use according to any one of (63) to (70), wherein agonizing β₃-adrenergic receptor comprises agonizing β₃-adrenergic receptor in the cardiovascular system of the subject.
(72) Vibegron for use according to any one of (63) to (71), wherein agonizing β₃-adrenergic receptor comprises agonizing β₃-adrenergic receptor in the myocardium of the subject.
(73) Vibegron for use according to any one of (63) to (72), wherein the vibegron is to be administered orally.
(74) Vibegron for use according to any one of (63) to (72), wherein the vibegron is to be administered intravenously.
(75) Vibegron for use according to any one of (63) to (74), wherein the therapeutically effective amount of vibegron is from about 50 mg to about 450 mg per day.
(76) Vibegron for use according to (75), wherein the therapeutically effective amount of vibegron is from about 50 mg to about 300 mg per day.
(77) Vibegron for use according to (76), wherein the therapeutically effective amount of vibegron is from about 100 mg to about 200 mg per day.
(78) Vibegron for use according to any one of (63) to (77), wherein a second therapeutic agent is to be administered to the subject.
(79) Vibegron for use according to (78), wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker, a neprolysin inhibitor antagonist, an alpha-2 receptor agonist, and a combination thereof
(80) Vibegron for use according to any one of (63) to (79), wherein vibegron is to be administered once per day.
(81) Vibegron for use according to (80), wherein vibegron is administered with a meal.
(82) Vibegron for use according to (80), wherein vibegron is administered without a meal.
(83) Vibegron for use according to any one of (63) to (82), wherein vibegron is administered as a free base.
(84) Vibegron for use according to any one of (63) to (82), wherein vibegron is administered as a pharmaceutically acceptable salt thereof.

The aspects described herein are further detailed with reference to the examples shown below. These examples are provided for the purpose of illustration only and the aspects described herein should in no way be construed as being limited to these examples. Rather, the aspects should be construed to encompass any and all variations which become evident as a result of the teachings provided herein.

### EXAMPLES

### Example 1: Selectivity and Maximum Response of Vibegron and Mirabegron for β₃-Adrenergic Receptors

The potency and maximal effect of vibegron and mirabegron for β₃-adrenergic receptors and the selectivity for each β-adrenergic receptor were determined. Functional cellular assays for vibegron and mirabegron activity were performed using Chinese hamster ovary (CHO) cells expressing β₁- or β₂-ARs and human embryonic kidney (HEK) 293 cells expressing β₃-ARs. Cells were incubated with vibegron, mirabegron, or the appropriate control (isoproterenol for β₁ and β₃, procaterol for β₂). Responses were quantified using homogeneous time-resolved fluorescence of cyclic adenosine monophosphate. Half-maximal effective concentration (EC₅₀) and maximum response (Eₘₐₓ) values were determined by nonlinear least-squares regression analysis.

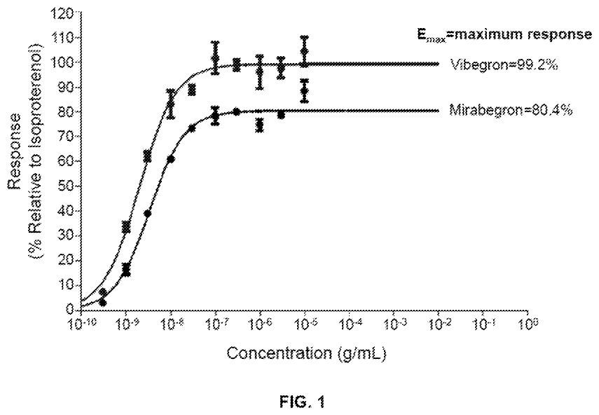

Treatment of HEK293 cells with vibegron or mirabegron resulted in concentration dependent responses at β₃-ARs (FIG. 1). Mean (SEM) EC₅₀ values at the β₃-AR were 2.13 (0.25) nM for vibegron and 10.0 (0.56) nM for mirabegron. At a concentration of 10 μM, β₃-adrenergic activity relative to isoproterenol was 104% for vibegron and 88% for mirabegron. In CHO cells, β₁-adrenergic activity was 0% and 3% for vibegron and mirabegron, respectively, and β₂-adrenergic activity was 2% and 15% for vibegron and mirabegron, respectively. Eₘₐₓ for vibegron and mirabegron at the β₃-AR was estimated to be 99.2% and 80.4%, for vibegron and mirabegron, respectively.

Table 1 shows the mean agonist activity at β₁-, β₂-, and β₃-adrenergic receptors relative to the full agonist controls isoproterenol (for β₁ and β₃) and procaterol (for β₂). As shown in the table, vibegron showed no measurable β₁ and low β₂ agonist activity compared with mirabegron, which showed measurable β₁ and some β₂ agonist activity. Both vibegron and mirabegron showed significant agonist activity at β₃-ARs as expected; however, vibegron demonstrated near-exclusive β₃ activity and was approximately 5 times more potent than mirabegron at activating β₃-ARs (mean EC₅₀ value at the β₃-AR was 2.13 nM for vibegron and 10.0 nM for miragegron). Vibegron also showed higher maximum β₃ response, approximating 100%.

TABLE 1
β-Receptor Selectivity

| | Vibegron | Mirabegron |
|---|---|---|
| Receptor | (% Activity) | (% Activity) |
| β₁ | 0 | 3 |
| β₂ | 2 | 15 |
| β₃ | 104 | 88 |

Without being bound by a particular theory, the superior selectivity of vibegron for the beta-3 adrenergic receptor suggests it can be effective in the treatment of heart failure.

### Example 2: Effect of Vibegron on Post-Myocardial Infarction (MI) Chronic Heart Failure (HF) in Mice

The purpose of this experiment is to study the effect of vibegron on structural remodeling (e.g., hypertrophy and/or fibrosis) and functional remodeling (e.g, ventricular function) in mice with chronic HF after a myocardial infarction (MI). The specific aims of these studies will be to determine the dose response treatment effects on cardiac remodeling by measuring parameters of cardiac structure and function using non-invasive, invasive, morphological, and histological assessments of mice after MI.

Myocardial infarction in the mouse is a commonly used experimental model for chronic ischemic heart failure. Due to complete ligation of the left anterior descending coronary artery, there is a large left ventricular infarct that produces adverse LV remodeling and significant dysfunction within 2 weeks post-MI. There also is significant fibrosis in this model and it is robust and reproducible with low mortality due to the surgical procedure. This model has been used for over 2 decades (see, e.g., Gao, E. et al., Circulation Research, 2010; 107: 1445-1453) and has been used to test potential novel HF therapeutics (Schumacher, S. et al. Science Translational Medicine, 2015; 7: 277).

Myocardial infarction was induced in 10-12 week C57B/6 wild-type mice and after 2 weeks echo cardiograms were used to assess the MI severity in each mouse. Mice were randomized for treatment based on similarity and severity of the induced infarct. The mice were started with either vibegron or vehicle (1-3 doses) administered by continuous infusion using 2 week minipumps (Azlet Corp.) that were filled with the appropriate dose and implanted subcutaneously on the back of each animal in the region of the scapula. The minipumps were replaced with new minipumps in the same location at the 2 week time point and dosed for 4 weeks in total. Cardiac function was assessed by echocardiography (Echo) at 0, 2, 4, and 6 weeks. Some mice were subjected to terminal catheterization to measure hemodynamic pressures and adrenergic responsiveness. At the end of the 4 week treatment period (i.e., 6 weeks after MI was induced), cardiac function was measured, mice were sacrificed for histology of the heart, and terminal blood was collected to confirm the serum concentration of vibegron.

Sham surgery groups treated with vehicle and highest dose of vibegron were included as controls. One group of post-MI mice treated with vehicle was also included in the study as a control. Two groupings of the 6 conditions were tested to allow for multiple replicates. There were 7 to 8 mice per condition in each group. The 2 groups studied were staggered by 3-4 weeks.

### Specific Protocol

Between 10 and 20 mice were first tested with vibegron in osmotic pumps for one week to determine serum concentrations released from pumps with specific buffers.

At this point, 6 experimental conditions (A-F below)×2 groups (staggered for reproducibility) were tested. Vibegron was provided in a form dissolved and delivered via an osmotic minipump placed under the skin of the mouse. The vehicle was used for negative control animals. Three (3) doses of vibegron (low, medium and high) were tested.

### Group 1 (7 or 8 Mice per Group)

A. Sham—vehicle
B. Sham—vibegron (high dose)
C. MI—vehicle
D. MI—vibegron—low dose
E. MI—vibegron—medium dose
F. MI—vibegron—high dose

### Group 2 (7 or 8 Mice per Group)—Repeated 3-4 Weeks after Group 1

A. Sham—vehicle
B. Sham—vibegron (high dose)
C. MI—vehicle
D. MI—vibegron—low dose
E. MI—vibegron—medium dose
F. MI—vibegron—high dose

### Schedule

1. Day 0 (after 1-2 weeks acclimation)—pre-surgery Echo
2. Perform LAD Ligation (MI) or Sham procedure
3. Day 14—Echo and randomly assign groups
4. Day 14—begin administration of vehicle and drug for 4 weeks
5. Day 28—Echo (operator blinded to groups)—change pumps for another 2 week dosing
6. Day 42—Echo (operator blinded to groups)
7. Day 42—Some were used for hemodynamics and ISO dose response and all animals sacrificed. Some were used for histology and some for RNA/Protein analysis. Blood was also collected at the time of sacrifice to determine serum concentrations.

### Methods

Surgical Methods: As described in Gao, E. et al., Circulation Research, 2010; 107: 1445-1453 and Schumacher, S. et al. Science Translational Medicine, 2015; 7: 277, mice were anesthetized and complete ligation of the LAD was performed through a partial thoracotomy. Following this, the mice were allowed to recover.

Non-Invasive Measurements: Echocardiography is a non-invasive, non-radioactive, painless imaging technique widely used in animals and humans. It operates on principles similar to sonar and consists of using ultrasound to create two-dimensional images of internal organs, specifically the heart with minimal stress on the mice. In these mouse MI studies, mice had serial echocardiograms as described in the protocols above.

Invasive Measurements: The hemodynamic measurements occurred at the end of the study as described in the protocols above. Again, according to the institutional IACUC protocol, the mice were anesthetized; the carotid artery was isolated and cannulated with a Millar pressure catheter. Hemodynamic measurements were collected and analyzed for cardiac function.

At the end of the study period, blood samples were processed for subsequent analysis of compound and decided biomarkers. At the end of the study, the heart was either preserved in formalin (for section, and staining) or snap frozen (for isolation of whole heart RNA, DNA and protein) as described above.

### Readouts

Cardiac Function (Echocardiography and hemodynamic pressure measurement at end of study):
Left ventricular pressures at baseline and after isoproterenol
Echocardiography: Left ventricular (LV) mass, LV chamber size, LV volumes, ejection fraction (EF) and fractional shortening (FS)
Systolic Arterial blood pressure
Determine heart rate (HR)

### Cardiac Structure (Morphology and Histology)

Heart weight/body weight or heart weight/tibia length, cardiac morphology (chamber length and size), fibrosis, myocyte size, inflammation

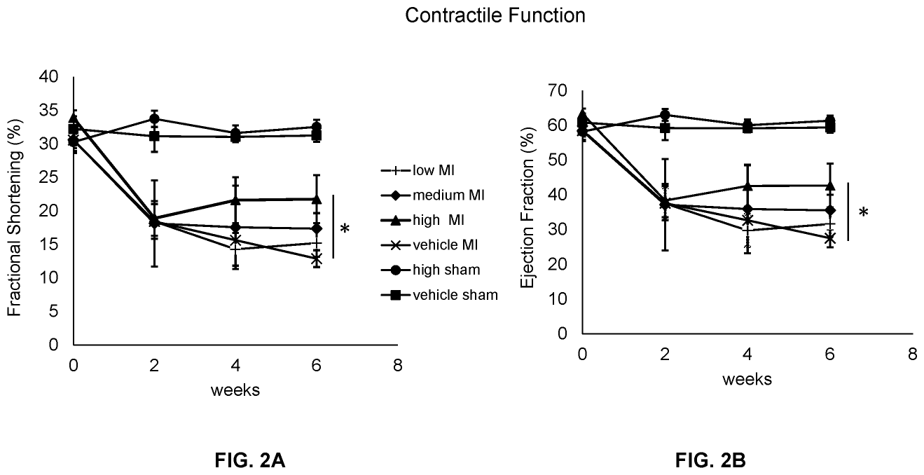

The contractile functions of fractional shortening (movement of the left ventricle wall) and ejection fraction (percentage of blood pumped out during systole) were measured, and the results are shown in Table 2 and FIGS. 2A and 2B, respectively. In this experiment, low dose refers to 1 milligram per kilogram (mg/kg) of vibegron by bodyweight, medium dose refers to 3 mg/kg and high dose refers to 10 mg/kg. Vehicle refers to a blank solution containing no vibegron and sham refers to a replicated surgery but with no myocardial infarction (MI) induced.

TABLE 2
Change in Contractile Functions Over Time

| | | | | | | |
|---|---|---|---|---|---|---|
| | Low Dose | Medium | High Dose | | High Dose | Vehicle |
| Contractile | of | Dose of | of | Vehicle | Vibegron - | Only - |
| Function | Vibegron | Vibegron | Vibegron | Only | Sham | Sham |

Baseline

| | | | | | | |
|---|---|---|---|---|---|---|
| Average % | 31.22 | 30.07 | 32.99 | 31.12 | 30.87 | 31.53 |
| Fractional | (0.58) | (1.13) | (0.90) | (1.23) | (0.55) | (1.09) |
| Shortening | | | | | | |
| (Standard | | | | | | |
| Error) | | | | | | |
| Average % | 59.50 | 58.11 | 62.06 | 59.23 | 59.04 | 59.80 |
| Ejection | (0.87) | (1.60) | (1.30) | (1.83) | (0.81) | (1.52) |
| Fraction | | | | | | |
| (Standard | | | | | | |
| Error) | | | | | | |

Week 2

| | | | | | | |
|---|---|---|---|---|---|---|
| Average % | 18.44 | 18.14 | 18.91 | 18.44 | 33.26 | 31.71 |
| Fractional | (2.55) | (6.41) | (2.59) | (2.61) | (0.84) | (0.97) |
| Shortening | | | | | | |
| (Standard | | | | | | |
| Error) | | | | | | |
| Average % | 37.52 | 37.18 | 38.39 | 37.62 | 62.36 | 60.07 |
| Ejection | (4.56) | (13.14) | (4.78) | (4.93) | (1.17) | (1.44) |
| Fraction | | | | | | |
| (Standard | | | | | | |
| Error) | | | | | | |

Week 4

| | | | | | | |
|---|---|---|---|---|---|---|
| Average % | 14.29 | 17.57 | 21.61 | 15.63 | 32.32 | 32.11 |
| Fractional | (2.45) | (6.21) | (3.41) | (1.90) | (0.82) | (0.70) |
| Shortening | | | | | | |
| (Standard | | | | | | |
| Error) | | | | | | |
| Average % | 29.77 | 35.95 | 42.59 | 32.69 | 61.08 | 60.78 |
| Ejection | (4.63) | (12.71) | (5.95) | (3.73) | (1.18) | (1.05) |
| Fraction | | | | | | |
| (Standard | | | | | | |
| Error) | | | | | | |

Week 6

| | | | | | | |
|---|---|---|---|---|---|---|
| Average % | 15.21 | 17.36 | 21.76 | 12.88 | 32.45 | 31.56 |
| Fractional | (2.04) | (2.32) | (3.59) | (1.25) | (0.82) | (0.59) |
| Shortening | | | | | | |
| (Standard | | | | | | |
| Error) | | | | | | |
| Average % | 31.64 | 35.55 | 42.68 | 27.54 | 61.18 | 59.83 |
| Ejection | (3.79) | (4.52) | (6.32) | (2.58) | (1.14) | (0.90) |
| Fraction | | | | | | |
| (Standard | | | | | | |
| Error) | | | | | | |

Table 2 and FIG. 2A show the change in fractional shortening (%) over a 6 week period. The results show a dose dependent correlation in fractional shortening as well as an overall improved fractional shortening in comparison with the vehicle treated mice after 4 weeks of vibegron treatment (i.e., at week 6) following induced MI. Moreover, mice dosed with the highest amount of vibegron showed improved fractional shortening at week 4 (i.e., 2 weeks after dosing).

Table 2 and FIG. 2B show the change in ejection fraction (%) over a 6 week period. The results again show a dose dependent correlation in improved ejection fraction as well as an overall improved ejection fraction in comparison with the vehicle treated mice after 4 weeks of vibegron treatment following induced MI. As with fractional shortening, mice dosed with the highest amount of vibegron showed an improvement in ejection fraction at week 4 (i.e., 2 weeks after dosing). As reduced fractional shortening has been seen in HFpEF (see, for example, J. Cardiovasc. Pharmacol. vol. 51, 1 Jan. 2008, pages 62-70) this data supports the use of vibegron in treating both HFrEF and HFpEF.

For both fractional shortening and ejection fraction, the sham mice treated with a high dose of vibegron and the sham mice treated with the solution only vehicle showed little to no deviation from their original baseline function, indicating no adverse effects were caused from either the operation or vibegron in healthy hearts.

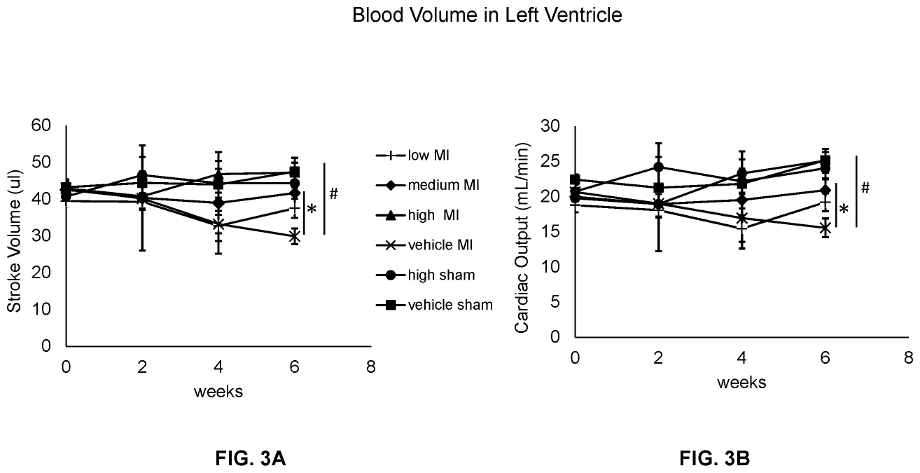

The left ventricle blood volumes were measured by stroke volume (microliters (μl) of blood pumped out during systole) and cardiac output (milliliters of blood pumped out per minute (mL/min)), and the results are shown in Table 3 and FIGS. 3A and 3B respectively. FIG. 3A and 3B both show a dose dependent correlation in increasing stroke volume and cardiac output, respectively, after 4 weeks of vibegron treatment in the MI induced mice, as well as overall increased left ventricle blood volumes in comparison with the vehicle treated mice, which showed no improvement in either stroke volume or cardiac output after 4 weeks of treatment following induced MI. Moreover, mice dosed with the highest amount of vibegron showed an improvement in stroke volume and cardiac output at week 4 (i.e., 2 weeks after dosing). A dose-response relationship in stroke volume and cardiac output at week 4 (i.e., 2 weeks after dosing) was demonstrated. As stroke volume and cardiac output are compromised in both HFrEF and HFpEF, this data supports the use of vibegron in treating both HFrEF and HFpEF.

TABLE 3
Change in Blood Volume in Left Ventricle Over Time

| | | | | | | |
|---|---|---|---|---|---|---|
| | Low | Medium | High Dose | | High Dose | Vehicle |
| | Dose of | Dose of | of | Vehicle | Vibegron - | Only - |
| Measurement | Vibegron | Vibegron | Vibegron | Only | Sham | Sham |

Baseline

| | | | | | | |
|---|---|---|---|---|---|---|
| Stroke | 42.74 | 44.72 | 43.30 | 44.64 | 42.25 | 45.28 |
| Volume (μL) | (1.88) | (2.19) | (1.58) | (2.45) | (1.37) | (1.78) |
| (Standard | | | | | | |
| Error) | | | | | | |
| Cardiac | 20.00 | 21.05 | 20.56 | 21.20 | 22.09 | 21.95 |
| Output | (0.94) | (1.42) | (1.03) | (0.88) | (0.84) | (1.05) |
| (mL/min) | | | | | | |
| (Standard | | | | | | |
| Error) | | | | | | |

Week 2

| | | | | | | |
|---|---|---|---|---|---|---|
| Stroke | 39.30 | 40.37 | 40.79 | 40.21 | 45.57 | 46.68 |
| Volume (μL) | (2.12) | (14.27) | (3.08) | (3.07) | (3.31) | (1.77) |
| (Standard | | | | | | |
| Error) | | | | | | |
| Cardiac | 18.06 | 18.94 | 18.93 | 19.00 | 23.72 | 23.59 |
| Output | (1.03) | (6.69) | (1.41) | (1.70) | (1.87) | (1.89) |
| (mL/min) | | | | | | |
| (Standard | | | | | | |
| Error) | | | | | | |

Week 4

| | | | | | | |
|---|---|---|---|---|---|---|
| Stroke | 32.76 | 39.00 | 46.83 | 33.25 | 44.44 | 42.69 |
| Volume (μL) | (4.09) | (13.79) | (3.65) | (2.54) | (2.11) | (1.52) |
| (Standard | | | | | | |
| Error) | | | | | | |
| % | — | 17.29 | 40.84 | — | — | — |
| Improvement | | | | | | |
| Relative to | | | | | | |
| Vehicle | | | | | | |
| Cardiac | 15.45 | 19.51 | 23.29 | 16.95 | 22.53 | 22.94 |
| Output | (1.89) | (6.90) | (1.97) | (1.35) | (1.06) | (1.00) |
| (mL/min) | | | | | | |
| (Standard | | | | | | |
| Error) | | | | | | |
| % | — | 15.10 | 37.40 | — | — | — |
| Improvement | | | | | | |
| Relative to | | | | | | |
| Vehicle | | | | | | |

Week 6

| | | | | | | |
|---|---|---|---|---|---|---|
| Stroke | 37.54 | 41.69 | 47.22 | 29.91 | 45.97 | 47.46 |
| Volume (μL) | (2.63) | (3.45) | (4.02) | (2.14) | (1.94) | (1.50) |
| (Standard | | | | | | |
| Error) | | | | | | |
| % | 25.51 | 39.38 | 57.87 | — | — | — |
| Improvement | | | | | | |
| Relative to | | | | | | |
| Vehicle | | | | | | |
| Cardiac | 19.19 | 20.90 | 25.09 | 15.55 | 23.63 | 25.03 |
| Output | (1.28) | (1.70) | (1.68) | (1.34) | (1.05) | (0.74) |
| (mL/min) | | | | | | |
| (Standard | | | | | | |
| Error) | | | | | | |
| % | 23.40 | 34.41 | 61.35 | — | — | — |
| Improvement | | | | | | |
| Relative to | | | | | | |
| Vehicle | | | | | | |

For both stroke volume and cardiac output, the sham mice treated with a high dose of vibegron and the sham mice treated with the solution only vehicle showed little to no deviation from their original baseline function, indicating no adverse effects were caused from either the operation or vibegron in healthy hearts.

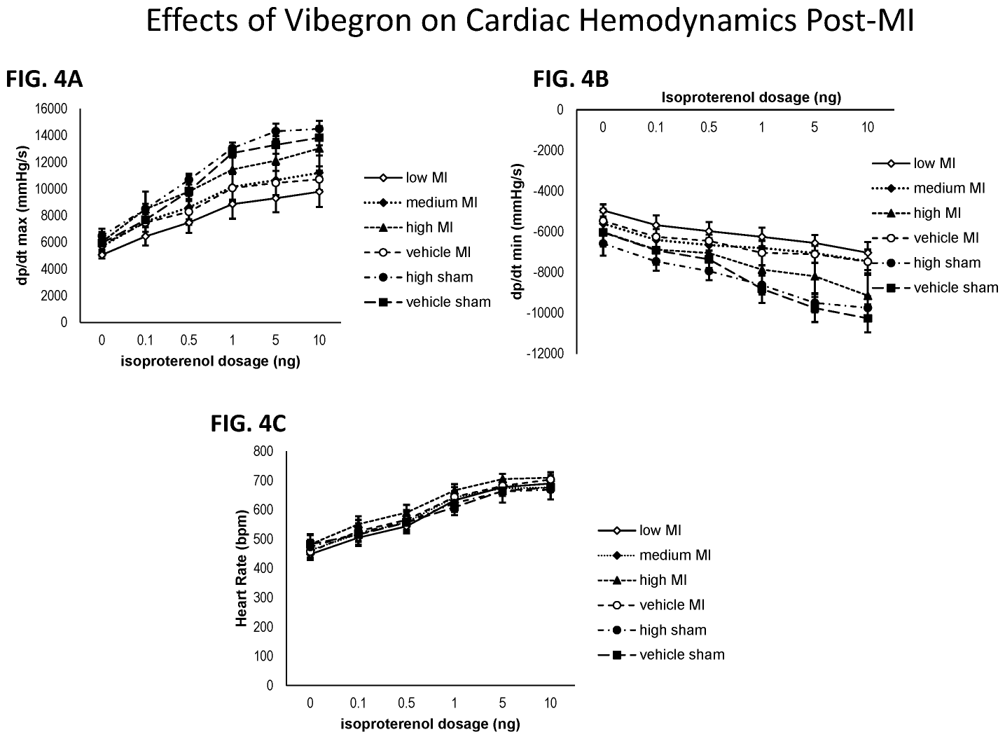

The maximum (dp/dt max) rate of pressure change during contraction and minimum (dp/dt min) rate of pressure change during relaxation of the left ventricle were measured with increasing doses of isoproterenol in combination with vibegron. The results are shown in FIGS. 4A and 4B, respectively.

As shown in FIG. 4A the maximum rate of pressure change with contraction increased after treatment with vibegron, indicating that vibegron improved blood flow. Similar results can be seen in FIG. 4B, which shows that treatment with vibegron decreased the minimum rate of pressure change during relaxation of the left ventricle. These results further confirm that treatment with vibegron improves blood flow in mice with induced myocardial infarctions.

In both measures of cardiac hemodynamics (FIGS. 4A and 4B), the effects were dose dependent, with higher doses of vibegron providing a greater improvement.

As demonstrated in FIG. 4C, mice with induced MI that were treated with vibegron showed a higher increase in heart rate when treated with increasing doses of isoproterenol than mice not receiving vibegron.

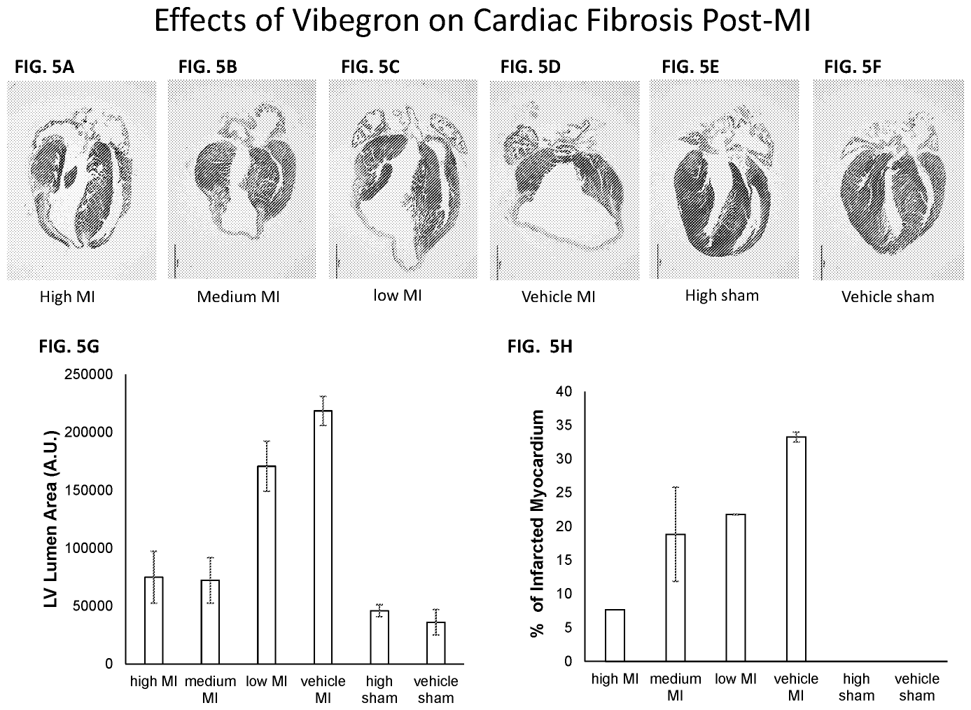

Histologies were obtained by sectioning the mouse hearts vertically in such a way that all four chambers were visible upon staining. The area of the left ventricle cavity was measured by staining with Mason's trichrome stain. Results are shown in FIGS. 5A-5F. The darker color indicates healthy myocardium and the lighter color indicates connective tissue, which is the largest component of infarcted myocardium. As shown in the figures, the amount of dark color was higher in the hearts of mice receiving vibegron than in the mice receiving only vehicle, indicating that treatment with vibegron increases the amount of healthy myocardium after infarction. These results were quantified as shown in Table 4 and FIGS. 5G and 5H.

TABLE 4
Heart Histology Results at Week 6

| | Low | Medium | High Dose | | High Dose | Vehicle |
|---|---|---|---|---|---|---|
| | Dose of | Dose of | of | Vehicle | Vibegron - | Only - |
| Measurement | Vibegron | Vibegron | Vibegron | Only | Sham | Sham |
| Average Left | 170,789 | 72,474 | 75,061 | 218,601 | 46,167 | 36,193 |
| Ventricle | (21,665) | (19,647) | (22,386) | (12,492) | (5,170) | (10,944) |
| Lumen Area | | | | | | |
| (AU) | | | | | | |
| (Standard | | | | | | |
| Error) | | | | | | |
| Average % | 21.79 | 18.85 | 7.68 | 33.27 | N/A | N/A |
| Infarcted | (0.04) | (6.97) | (7.68) | (0.74) | | |
| Myocardium | | | | | | |
| (Standard | | | | | | |
| Error) | | | | | | |

FIG. 5G shows the area of LV lumen, which increases after myocardial infarction, in mice treated with varying amounts of vibegron in comparison to mice treated with vehicle. As shown in the figure, mice treated with vibegron had smaller amounts of LV lumen than mice treated with vehicle.

FIG. 5H shows the percentage of infarcted myocardium after 4 weeks of treatment with vibegron. As shown in the figure, mice treated with vibegron had a lower percentage of infarcted myocardium than mice treated with vehicle. In addition, mice treated with the highest dose of vibegron had the lowest percentage of infarcted myocardium of any of the treated mice.

The sham mice treated with a high dose of vibegron and the sham mice treated with the solution only vehicle had the smallest LV lumen area and also had no infarcted myocardium, confirming no adverse effects were caused from either the operation or vibegron in healthy hearts.

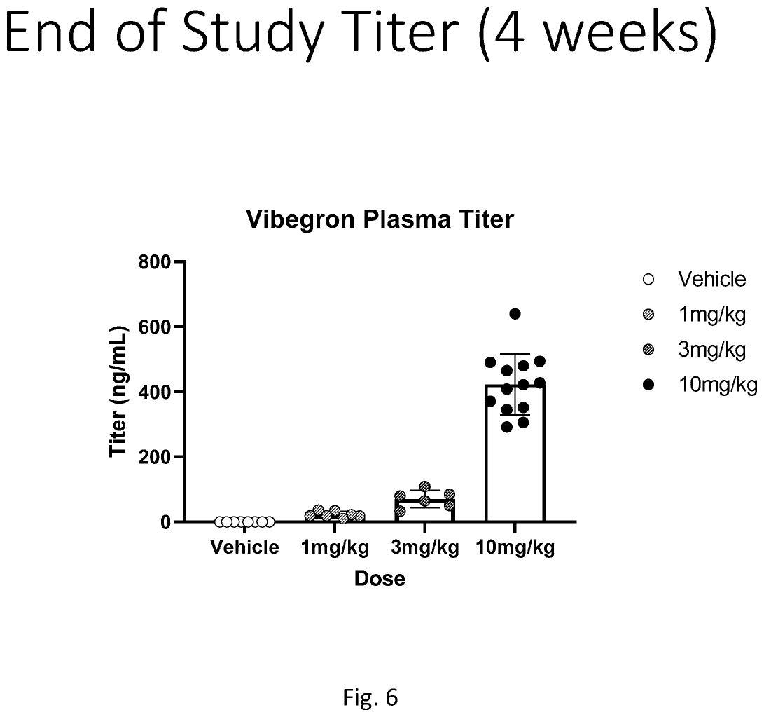

A plasma titer was performed following the 4 weeks of treatment (i.e., at week 6) after induced myocardial infarction. Results are shown in FIG. 6. The results show that after 4 weeks of treatment, the presence of vibegron in the mouse plasma was consistent with the dosing. Mice receiving the lowest doses of vibegron showed lower presence of vibegron than mice dosed with the medium and high dosages. As expected, the vehicle treated mice showed no presence of vibegron in the plasma titer.

### Example 3: Clinical Study of Vibegron in Patients with Heart Failure

The objective of this study will be to study the cardiac effects of treatment with vibegron in patients diagnosed with class HF (according to the New York Heart Association (NYHA) standard) currently receiving standard of care treatment. Patients will be administered a dose of vibegron daily and titrated up to a maximum tolerated dose. After three months, the following outcomes will be measured:
increase in fractional shortening (as measured by echocardiogram);
decrease in level of biomarker NT proBNP; and
increase in 6 min walking distance.

Outcomes for patients with HFrEF (less than about 40% LVEF), HFpEF (greater than about 50% LVEF) and HFmrEF (40%-50% LVEF) will be compared.

If results are statistically inconclusive, the study can optionally be extended for an additional three months or additional patients can be added.

Having now fully described this invention, it will be understood by those of ordinary skill in the art that the same can be performed within a wide and equivalent range of conditions, formulations and other parameters without affecting the scope of the invention or any aspect thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, and other publications cited herein are fully incorporated by reference herein in their entirety.

What is claimed is:

1. A method for treating or preventing heart failure with preserved ejection fraction (HFpEF), one or more symptoms thereof, or both in a subject in need thereof, the method comprising administering a therapeutically effective amount of vibegron to the subject.

2. The method of claim 1, wherein the subject has a left ventricular ejection fraction of greater than about 40%.

3. The method of claim 1 or 2, wherein the subject has a left ventricular ejection fraction of greater than 50%.

4. The method of any one of claims 1 to 3, wherein the therapeutically effective amount of vibegron is from about 90 mg to about 450 mg per day.

5. The method of claim 4, wherein the therapeutically effective amount of vibegron is from about 100 mg to about 400 mg per day.

6. The method of claim 5, wherein the therapeutically effective amount of vibegron is from about 100 mg to about 200 mg per day.

7. The method of any one of claims 1 to 6, further comprising administering to the subject a second therapeutic agent.

8. The method of claim 7, wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker, a neprolysin inhibitor antagonist, an alpha-2 receptor agonist, and a combination thereof.

9. The method of any one of claims 1 to 8, wherein the vibegron is administered orally.

10. The method of any one of claims 1 to 9, wherein the vibegron is administered intravenously.

11. The method of any one of claims 1 to 10, wherein the subject receiving vibegron experiences a smaller maximum mean change in systolic blood pressure than a subject receiving a therapeutically effective amount of mirabegron.

12. The method of any one of claims 1 to 11, wherein the subject receiving vibegron experiences an increase in maximum mean change in systolic blood pressure of from about 1.5 mm Hg to about 4.0 mm Hg smaller than a subject receiving a therapeutically effective amount of mirabegron.

13. The method of any one of claims 1 to 12, wherein the subject receiving vibegron experiences a smaller mean 24 hour change in systolic blood pressure than a subject receiving a therapeutically effective amount of mirabegron.

14. The method of any one of claims 1 to 13, wherein the subject receiving vibegron experiences an increase in mean 24 hour change in systolic blood pressure of from about 1.0 mm Hg to about 10.0 mm Hg smaller than a subject receiving a therapeutically effective amount of mirabegron.

15. The method of any one of claims 1 to 14, wherein the subject receiving vibegron experiences a smaller maximum increase in heart rate than a subject receiving a therapeutically effective amount of mirabegron.

16. The method of any one of claims 1 to 15, wherein the subject receiving vibegron experiences an increase in maximum mean increase in heart rate that is from about 2 bpm to about 14 bpm smaller than a subject receiving a therapeutically effective amount of mirabegron.

17. The method of any one of claims 11 to 16, wherein the therapeutically effective amount of mirabegron is from about 50 mg to about 300 mg.

18. The method of any one of claims 1 to 17, wherein treating comprises halting the progression of HFpEF, relieving one or more symptoms associated with HFpEF, or both.

19. A method for treating or preventing heart failure with reduced ejection fraction (HFrEF), one or more symptoms thereof, or both, in a subject in need thereof, the method comprising administering a therapeutically effective amount of vibegron to the subject.

20. The method of claim 19, wherein the subject has a left ventricular ejection fraction less than 50%.

21. The method of claim 19 or 20, wherein the subject has a left ventricular ejection fraction of less than about 40%.

22. The method of any one of claims 19 to 21, wherein the therapeutically effective amount of vibegron is from about 90 mg to about 450 mg per day.

23. The method of claim 22, wherein the therapeutically effective amount of vibegron is from about 100 mg to about 400 mg per day.

24. The method of claim 23, wherein the therapeutically effective amount of vibegron is from about 100 mg to about 200 mg per day.

25. The method of any one of claims 19 to 24, further comprising administering to the subject a second therapeutic agent.

26. The method of claim 25, wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker, a neprolysin inhibitor antagonist, an alpha-2 receptor agonist, and a combination thereof.

27. The method of any one of claims 19 to 26, wherein the vibegron is administered orally.

28. The method of any one of claims 19 to 26 wherein the vibegron is administered intravenously.

29. The method of any one of claims 19 to 28, wherein the subject receiving vibegron experiences a smaller maximum mean change in systolic blood pressure than a subject receiving a therapeutically effective amount of mirabegron.

30. The method of any one of claims 19 to 29, wherein the subject receiving vibegron experiences an increase in maximum mean change in systolic blood pressure of from about 1.5 mm Hg to about 4.0 mm Hg smaller than a subject receiving a therapeutically effective amount of mirabegron.

31. The method of any one of claims 19 to 30, wherein the subject receiving vibegron experiences a smaller mean 24 hour change in systolic blood pressure than a subject receiving a therapeutically effective amount of mirabegron.

32. The method of any one of claims 19 to 31, wherein the subject receiving vibegron experiences an increase in mean 24 hour change in systolic blood pressure of from about 1.0 mm Hg to about 10.0 mm Hg smaller than a subject receiving a therapeutically effective amount of mirabegron.

33. The method of any one of claims 19 to 32, wherein the subject receiving vibegron experiences a smaller maximum increase in heart rate than a subject receiving a therapeutically effective amount of mirabegron.

34. The method of any one of claims 19 to 33, wherein the subject receiving vibegron experiences an increase in maximum mean increase in heart rate that is from about 2 bpm to about 14 bpm smaller than a subject receiving a therapeutically effective amount of mirabegron.

35. The method of any one of claims 30 to 34, wherein the therapeutically effective amount of mirabegron is from about 50 mg to about 300 mg.

36. The method of any one of claims 19 to 35, wherein treating comprises halting the progression of HFrEF, relieving one or more symptoms associated with HFrEF, or both.

37. The method of any one of claims 1 to 36, wherein vibegron is administered once per day.

38. The method of claim 37, wherein vibegron is administered with a meal.

39. The method of claim 38, wherein vibegron is administered without a meal.

40. The method of any one of claims 1 to 39, wherein vibegron is administered as a free base.

41. The method of any one of claims 1 to 39, wherein vibegron is administered as a pharmaceutically acceptable salt thereof.

42. The method of any one of claims 1 to 41, wherein the subject's contractile function is increased or improved relative to a subject who has not received vibegron or as compared to the subject's contractile function before receiving vibegron.

43. The method of claim 42, wherein the increase or improvement in the subject's contractile function is measured by fractional shortening.

44. The method of claim 43, wherein the subject's fractional shortening is increased relative to a subject who has not received vibegron.

45. The method of claim 44, wherein the subject's fractional shortening is increased by about 2% to about 15% more than a subject who has not received vibegron.

46. The method of claim 45, wherein the subject's fractional shortening is increased by about 5% to about 10% more than a subject who has not received vibegron.

47. The method of claim 42, wherein the increase or improvement in the subject's contractile function is measured by ejection fraction.

48. The method of claim 47, wherein the subject's ejection fraction is increased relative to a subject who has not received vibegron.

49. The method of claim 48, wherein the subject's ejection fraction is increased by about 2% to about 20% more than a subject who has not received vibegron.

50. The method of claim 49, wherein the subject's ejection fraction is increased by about 10% to about 20% more than a subject who has not received vibegron

51. The method of any one of claims 1 to 50, wherein blood volume in the subject's left ventricle is increased or improved relative to a subject who has not received vibegron or as compared to the subject's blood volume before receiving vibegron.

52. The method of claim 51, wherein the increase or improvement in the subject's blood volume is measured by stroke volume in the subject's left ventricle.

53. The method of claim 52, wherein stroke volume in the subject's left ventricle is increased relative to a subject who has not received vibegron.

54. The method of claim 53, wherein the stroke volume in the subject's left ventricle is increased by about 25% to about 75% relative to a subject who has not received vibegron.

55. The method of claim 54, wherein the stroke volume in the subject's left ventricle is increased by about 30% to about 65% relative to a subject who has not received vibegron.

56. The method of claim 49, wherein the increase or improvement in the subject's blood volume is measured by cardiac output.

57. The method of claim 56, wherein the subject's cardiac output is increased relative to a subject who has not received vibegron.

58. The method of claim 57, wherein the subject's cardiac output is increased by about 25% to about 75% relative to a subject who has not received vibegron.

59. The method of claim 58, wherein the subject's cardiac output is increased by about 30% to about 65% relative to a subject who has not received vibegron.

60. The method of any one of claims 1 to 59, wherein vibegron has an onset of action of about 4 weeks, about 3 weeks, or about 2 weeks.

61. The method of claim 60, wherein vibegron has an onset of action within about 2 weeks.

62. The method of any one of claims 1 to 61, wherein the subject experiences one or more of: a shortened duration of hospital stay, an increase in the number of days between hospital stays, a reduced risk of hospitalization, an increase in the time before an initial hospitalization, or a fewer number of hospitalizations relative to a subject who has not received vibegron or as compared to the subject's experience before receiving vibegron.

63. A method of agonizing β₃-adrenergic receptor and antagonizing β₁-adrenergic receptor, β₂-adrenergic receptor, or both β₁-adrenergic receptor and β₂-adrenergic receptor in a subject in need thereof, the method comprising administering
(a) a therapeutically effective amount of vibegron to the subject to agonize β₃-adrenergic receptor and
(b) a therapeutically effective amount of a therapeutic agent that antagonizes β₁-adrenergic receptor, that antagonizes β₂-adrenergic receptor, or that antagonizes both β₁-adrenergic receptor and β₂-adrenergic receptor.

64. A method of agonizing β₃-adrenergic receptor in a subject in need thereof, the method comprising administering a therapeutically effective amount of vibegron to the subject.

65. The method of claim 63 or 64, wherein the subject suffers from heart failure, one or more symptoms thereof, or both.

66. The method of any one of claims 63 to 65, wherein the subject has heart failure with preserved ejection fraction (HFpEF), one or more symptoms thereof, or both.

67. The method of any one of claims 63 to 65, wherein the subject has heart failure with reduced ejection fraction (HFrEF), one or more symptoms thereof, or both.

68. The method of any one of claims 63 to 65, wherein the subject has heart failure with mid-range ejection fraction (HFmrEF), one or more symptoms thereof, or both.

69. The method of any one of claims 63 to 66, wherein agonizing β₃-adrenergic receptor comprises agonizing β₃-adrenergic receptor in the cardiovascular system of the subject.

70. The method of any one of claims 63 to 67, wherein agonizing β₃-adrenergic receptor comprises agonizing β₃-adrenergic receptor in the myocardium of the subject.

71. The method of any one of claims 63 to 70, wherein the vibegron is administered orally.

72. The method of any one of claims 63 to 70, wherein the vibegron is administered intravenously.

73. The method of any one of claims 63 to 72, wherein the therapeutically effective amount of vibegron is from about 50 mg to about 450 mg per day.

74. The method of claim 73, wherein the therapeutically effective amount of vibegron is from about 50 mg to about 300 mg per day.

75. The method of claim 74, wherein the therapeutically effective amount of vibegron is from about 100 mg to about 200 mg per day.

76. The method of any one of claims 63 to 72, further comprising administering to the subject a second therapeutic agent.

77. The method of claim 76, wherein the second therapeutic agent is selected from an antihyperlipoproteinemic agent, an antiarteriosclerotic agent, an antithrombotic/fibrinolytic agent, a blood coagulant, an antiarrhythmic agent, an antihypertensive agent, a vasopressor, a diuretic, a statin, an anti-platelet agent, an SGLT2 inhibitor, a treatment agent for congestive heart failure, an antianginal agent, an antibacterial agent, a renin-angiotensin system (RAS) blocker, a mineralocorticoid receptor antagonist (MRA), an ACE inhibitor, an angiotensin receptor blocker, a neprolysin inhibitor antagonist, an alpha-2 receptor agonist, and a combination thereof.

78. The method of any one of claims 63 to 77, wherein vibegron is administered once per day.

79. The method of claim 78, wherein vibegron is administered with a meal.

80. The method of claim 79, wherein vibegron is administered without a meal.

81. The method of any one of claims 63 to 80, wherein vibegron is administered as a free base.

82. The method of any one of claims 63 to 80, wherein vibegron is administered as a pharmaceutically acceptable salt thereof.
